(19) United States
(12) Patent Application Publication
(10) Pub. No.: US 2024/0156832 A1
(43) Pub. Date: May 16, 2024

(54) METHODS OF METABOLIZING METOPIMAZINE AND ITS SALTS

(71) Applicant: Neurogastrx, Inc., Woburn, MA (US)

(72) Inventors: Robert W. Busby, Woburn, MA (US); Cyril De Colle, Woburn, MA (US)

(21) Appl. No.: 18/270,001

(22) PCT Filed: Dec. 28, 2021
(86) PCT No.: PCT/US2021/065290

§ 371 Date: Jun. 28, 2023

Related U.S. Application Data
(60) Provisional application No. 63/131,419, filed on Dec. 29, 2020.
(60) Provisional application No. 63/219,047, filed on Jul. 7, 2021.

(51) Int. Cl. A61K 31/5415; C12P 17/18

(52) U.S. Cl. CPC A61K 31/5415; C12P 17/185

(57) ABSTRACT

Provided herein is process of preparing metopimazine acid by reacting metopimazine, or a pharmaceutically acceptable salt thereof, with human microsomal liver amidase. Also provided herein are methods of treating gastroparesis in a human subject in need thereof with metopimazine, or a pharmaceutically acceptable salt thereof, in combination with an additional therapeutic agent which is not metabolized by human microsomal liver amidase and/or human liver cytosolic aldehyde oxidase.

### RELATED APPLICATIONS

This application claims the benefit of priority to U.S. Provisional Patent Application No. 63/131,419, filed Dec. 29, 2020, and U.S. Provisional Patent Application No. 63/219,047, filed Jul. 7, 2021, which applications are hereby incorporated by reference in their entirety.

### BACKGROUND

The enteric nervous system (ENS) comprises about one hundred million neurons embedded in the lining of the gastrointestinal system. The ENS innervates the gastrointestinal system, including the esophagus, the stomach (e.g., gastric area), and the intestines. Motor neurons of the ENS control stomach muscle contractility, peristalsis, and churning of intestinal contents. It has been estimated that about 50% of the body's dopamine is found in the ENS.

Gastrointestinal (GI) tract disorders affect many people. Irritable bowel syndrome (IBS), a disorder in which the intestine functions abnormally due to dysfunction of the muscles or nerves of the GI tract, affects 10 to 15% of the adult population. Symptoms of IBS include constipation, diarrhea, and abdominal pain. Functional dyspepsia (dyspepsia caused by a dysfunction of the muscles or nerves associated with the upper GI tract) affects 10 to 20% of the adult population. Gastroparesis, a disorder causing inadequate grinding of food by the stomach and delayed gastric emptying, affects up to 10% of the general population. Gastroesophageal reflux disorder (GERD), a chronic digestive disease that occurs when stomach acid and/or bile backs up into the esophagus, has been estimated to affect up to 35% of infants in the first few months of life and more than half of the general population in the US.

In addition, gastrointestinal disorders can be associated with a number of other diseases. For example, some of the earliest symptoms of Parkinson's disease, a disorder characterized by neurodegeneration of dopamine neurons, include, e.g., constipation and other gastrointestinal symptoms, likely due to degeneration or dysfunction of ENS dopamine neurons. Another example is diabetes, one of the most common causes of gastroparesis, as chronic high blood sugar can damage the vagus nerve which modulates the enteric nervous system. Multiple sclerosis is another disease that is associated with ENS disorders such as, e.g., gastroparesis. Migraine headaches are commonly associated with gastric stasis. Chemotherapy-induced nausea and/or vomiting have been estimated to affect 85% of cancer patients undergoing chemotherapy and can result in discontinuation of treatment. If the chemotherapy-induced nausea and/or vomiting are not properly managed, it can cause dehydration and poor quality of life and may result in discontinuation of chemotherapy.

ENS dysfunction has been implicated in several of the disorders described above. For example, impaired or dysfunctional ENS neuronal signaling has been strongly implicated as a causative factor for gastroparesis.

There are currently no adequate treatments for these disorders. For example, the IBS treatment lubiprostone and is used to mimic infectious diarrhea in order to treat constipation; however, this agent does not correct the underlying ENS dysfunction and is not universally effective. The dopamine D2 receptor antagonists domperidone and metoclopramide have been previously indicated for the treatment of nausea and vomiting, however, their use is discouraged due to significant safety issues, in particular for extended periods of time. Two significant safety concerns relate to (1) unwanted cardiac side effects caused by, e.g., interaction of the agents with ion channels involved in cardiac action potentials, and (2) unwanted motor dysfunction caused by the actions of the dopamine antagonists which cross the blood brain barrier into the brain. For example, it has been established that many dopamine receptor antagonists inhibit hERG channels (a type of potassium channel) to cause drug-induced long QT syndrome, a heart condition characterized by abnormal cardiac action potential rhythms. Long QT syndrome can increase risk of cardiac arrhythmias, which may lead to sudden cardiac death. Indeed, the dopamine D2 antagonist domperidone has been shown to inhibit hERG activity and increase risk of long QT syndrome, and increase risk of sudden cardiac death. This has resulted in an FDA ban on the use of domperidone in the United States and an initiated review of the safety of domperidone use by the European Medicines Agency. Metoclopramide cannot be taken for more than 12 weeks and has a black box warning for CNS-related side effects such as tardive dyskinesia, a difficult-to-treat and often incurable disorder characterized by involuntary, repetitive body movements.

Metopimazine (MPZ) is an approved drug in France under the brand name Vogalene® (metopimazine, free base) that has been used for over four decades for the short-term treatment of nausea and vomiting. A new salt formulation of MPZ is in clinical development in the US for the treatment of gastroparesis, a chronic disorder of the stomach characterized by delayed gastric emptying without evidence of mechanical obstruction (Camilleri et al. (2013) “Clinical Guideline: Management of Gastroparesis” Am. J. Gastroenterol, 108:18-38; Stein et al. (2015) “Gastroparesis A Review of Current Diagnosis and Treatment Options” J. Clin. Gastroenterol. 49:550-558; NCT04303195). MPZ is a phenothiazine (1) that is subject to high first pass metabolism in humans (Herrstedt et al., (1990) “The effect of food on serum concentrations of metopimazine” Br. J. Gin. Pharmacol. 30:237-43; Mallet, et al., (2015) “Pharmacokinetic study of metopimainze by oral route in children” Pharma. Res. Per., 3(3):1-7). MPZ is a potent D2/D3 selective, dopamine receptor antagonist that does not penetrate the blood brain barrier (De Colle et al., (2016) “A Potent and Selective Dopamine D2 Receptor Antagonist as a Potential Alternative to Metoclopramide and Domperidone for the Treatment of Gastroparesis” AGA Abstracts Volume 150, Issue 4, Supplement 1, S214). While much is known about MPZ, the metabolism of this drug is not fully elucidated. MPZ is deaminated to form metopimazine acid (MPZA) (2), the major circulating metabolite, present at much higher plasma concentrations than the parent (Herrstedt et al., 1990). MPZA retains some pharmacologic activity but is about 200-fold less potent at the human D2 receptor compared to the parent (De Colle et al., 2016). Given the renewed interest in exploring the potential of MPZ for new clinical indications, identifying the enzyme(s) responsible for the biotransformation of this drug into its primary metabolite is important to understanding any potential impact of metabolic variability on efficacy and safety risks and in assessing drug-drug interaction potential.

### SUMMARY OF THE APPLICATION

The present application provides a process of preparing metopimazine acid,

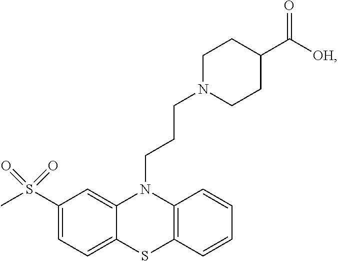 [SMILES: CS(=O)(=O)c1ccc2c(c1)N(CCCN1CCC(C(=O)O)CC1)c1ccccc1S2]

comprising contacting metopimazine,

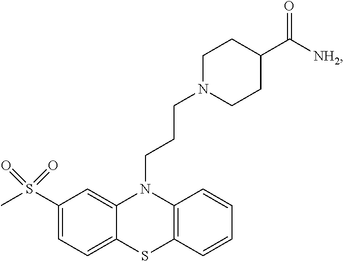 [SMILES: CS(=O)(=O)c1ccc2c(c1)N(CCCN1CCC(C(N)=O)CC1)c1ccccc1S2]

or a pharmaceutically acceptable salt thereof, with human microsomal liver amidase. The present application further provides a process of preparing metopimazine acid, comprising contacting metopimazine, or a pharmaceutically acceptable salt thereof, with human liver cytosolic aldehyde oxidase. In certain embodiments of the foregoing processes, the metopimazine, or a pharmaceutically acceptable salt thereof, is metopimazine mesylate,

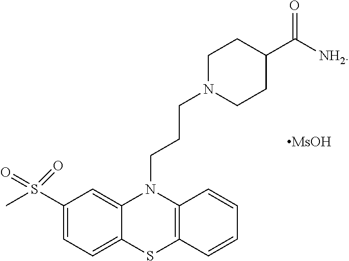 [SMILES: "·MsOH" ; CS(=O)(=O)c1ccc2c(c1)N(CCCN1CCC(C(N)=O)CC1)c1ccccc1S2]

The present application provides a method of treating gastroparesis in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase. The present application further provides a method of treating gastroparesis in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase. In certain embodiments of the foregoing methods, the gastroparesis is diabetic gastroparesis. In certain other embodiments of the foregoing methods, the gastroparesis is idiopathic gastroparesis. In certain embodiments, the gastroparesis comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the gastroparesis comprises the symptom nausea. In other such embodiments, the gastroparesis comprises the symptom vomiting.

The present application provides a method of treating nausea associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase. The present application further provides a method of treating nausea associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

The present application provides a method of treating vomiting associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase. The present application further provides a method of treating vomiting associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

The present application provides a method of improving gastric emptying in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase. The present application further provides a method of improving gastric emptying in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

The present application provides a method of treating functional and motility disorders of the GI tract in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase. The present application further provides a method of treating functional and motility disorders of the GI tract in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

The present application provides a method of treating an enteric nervous system disorder in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase. The present application further provides a method of treating an enteric nervous system disorder in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase. In certain embodiments, the enteric nervous system disorder is a chronic disorder. In other embodiments, the enteric nervous system disorder is an acute disorder. In certain embodiments, the enteric nervous system disorder is selected from the group consisting of gastroparesis, Irritable Bowel Syndrome, lysosomal storage disorders, intestinal dysmotility, ganglioneuroma, multiple endocrine neoplasia type 2B (MEN2B), gastrointestinal neuropathy, functional dyspepsia, gastroesophageal reflux disease (GERD), and intestinal neuronal dysplasia. In certain embodiments, the enteric nervous system disorder comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the enteric nervous system disorder comprises the symptom nausea. In other such embodiments, the enteric nervous system disorder comprises the symptom vomiting.

In certain embodiments of any of the foregoing methods, the metopimazine, or a pharmaceutically acceptable salt thereof, is administered to the subject chronically. In certain other embodiments of any of the foregoing methods, the metopimazine, or a pharmaceutically acceptable salt thereof, is administered to the subject acutely.

In certain embodiments of any of the foregoing methods, the metopimazine, or a pharmaceutically acceptable salt thereof, is administered to the subject for at least 6 days, for at least 7 days, for at least four weeks, or for at least 12 weeks.

In certain embodiments of any of the foregoing methods, the metopimazine, or a pharmaceutically acceptable salt thereof, is administered to the subject one time per day, two times per day, three times per day, or four times per day.

In certain embodiments of any of the foregoing methods, between about 5 mg and about 160 mg of the metopimazine, or a pharmaceutically acceptable salt thereof, is administered to the subject per day. In certain such embodiments, more than 20 mg of metopimazine, or a pharmaceutically acceptable salt thereof, is administered to the subject per day.

In certain embodiments of any of the foregoing methods, the metopimazine, or a pharmaceutically acceptable salt thereof, and the additional therapeutic agent are administered simultaneously in separate compositions. In certain other embodiments of any of the foregoing methods, the metopimazine, or a pharmaceutically acceptable salt thereof, and the additional therapeutic agent are administered at different times and in separate compositions. In certain other embodiments of any of the foregoing methods, the metopimazine, or a pharmaceutically acceptable salt thereof, and the additional therapeutic agent are administered in a composition in which both agents are present.

In certain embodiments of any of the foregoing methods, the metopimazine, or a pharmaceutically acceptable salt thereof, is suitable for administering orally, intraduodenally, intracolonically, parenterally, enterally, intraperitoneally, topically, transdermally, ophthalmically, intranasally, locally, non-orally, via spray, subcutaneously, intravenously, intratonsillary, intramuscularly, buccally, sublingually, intra-arterially, intrathecally, by infusion, by inhalation, or rectally, such as orally, intraduodenally, intracolonically, enterally, topically, intranasally, non-orally, buccally, sublingually, by inhalation, or rectally. In certain such embodiments, the metopimazine, or a pharmaceutically acceptable salt thereof, is suitable for administering orally. In certain other embodiments, the metopimazine, or a pharmaceutically acceptable salt thereof, is suitable for administering sublingually.

In certain embodiments of any of the foregoing methods, the metopimazine, or a pharmaceutically acceptable salt thereof, is formulated as a tablet, a capsulean oil, a capsule, a gel, a paste, a powder, a suspension, a syrup, an enema, a suppository, an emulsion, a solution, an extended-release formulation, or a modified-release formulation, such as formulated as a tablet, a capsule, a paste, a powder, a suspension, a suppository, an extended-release formulation, or a modified-release formulation. In certain such embodiments, the metopimazine, or a pharmaceutically acceptable salt thereof, is formulated as an extended-release formulation. In certain other embodiments, the metopimazine, or a pharmaceutically acceptable salt thereof, is formulated as a capsule.

In certain embodiments of any of the foregoing methods, the metopimazine, or a pharmaceutically acceptable salt thereof, is metopimazine mesylate.

The present application further provides a pharmaceutical composition comprising metopimazine, or a pharmaceutically acceptable salt thereof, in combination with an additional therapeutic agent, wherein the additional therapeutic agent is not metabolized by human microsomal liver amidase. The present application further provides a pharmaceutical composition comprising metopimazine, or a pharmaceutically acceptable salt thereof, in combination with an additional therapeutic agent, wherein the additional therapeutic agent is not metabolized by human liver cytosolic aldehyde oxidase. In certain embodiments of the foregoing pharmaceutical compositions, the metopimazine, or a pharmaceutically acceptable salt thereof, is a pharmaceutically acceptable salt of metopimazine. In certain such embodiments, the pharmaceutically acceptable salt of metopimazine is metopimazine mesylate.

The present application further provides a method of treating gastroparesis in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase. In certain embodiments, the gastroparesis is diabetic gastroparesis. In other embodiments, the gastroparesis is idiopathic gastroparesis. In certain embodiments of the foregoing, the gastroparesis comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the gastroparesis comprises the symptom nausea. In other such embodiments, the gastroparesis comprises the symptom vomiting.

A method of treating nausea associated with gastroparesis in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase. A method of treating vomiting associated with gastroparesis in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of improving gastric emptying in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of treating functional and motility disorders of the GI tract in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of treating an enteric nervous system disorder in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase. In certain embodiments, the enteric nervous system disorder is a chronic disorder. In other embodiments, the enteric nervous system disorder is an acute disorder. In certain embodiments of the foregoing, the enteric nervous system disorder is selected from the group consisting of gastroparesis, Irritable Bowel Syndrome, lysosomal storage disorders, intestinal dysmotility, ganglioneuroma, multiple endocrine neoplasia type 2B (MEN2B), gastrointestinal neuropathy, functional dyspepsia, gastroesophageal reflux disease (GERD), and intestinal neuronal dysplasia. In certain embodiments, the enteric nervous system disorder comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the enteric nervous system disorder comprises the symptom nausea. In other such embodiments, the enteric nervous system disorder comprises the symptom vomiting.

In certain embodiments of any of the foregoing methods, the pharmaceutical composition is administered to the subject chronically. In certain other embodiments of any of the foregoing methods, the pharmaceutical composition is administered to the subject acutely.

In certain embodiments of any of the foregoing methods, the pharmaceutical composition is administered to the subject for at least 6 days, for at least 7 days, for at least four weeks, or for at least 12 weeks.

In certain embodiments of any of the foregoing methods, the pharmaceutical composition is administered to the subject one time per day, two times per day, three times per day, or four times per day.

In certain embodiments of any of the foregoing methods, between about 5 mg and about 160 mg of the metopimazine, or a pharmaceutically acceptable salt thereof, is administered to the subject per day. In certain such embodiments, more than 20 mg of metopimazine, or a pharmaceutically acceptable salt thereof, is administered to the subject per day.

In certain embodiments of any of the foregoing methods, pharmaceutical composition is suitable for administering orally, intraduodenally, intracolonically, parenterally, enterally, intraperitoneally, topically, transdermally, ophthalmically, intranasally, locally, non-orally, via spray, subcutaneously, intravenously, intratonsillary, intramuscularly, buccally, sublingually, intra-arterially, intrathecally, by infusion, by inhalation, or rectally, such as orally, intraduodenally, intracolonically, enterally, topically, intranasally, non-orally, buccally, sublingually, by inhalation, or rectally. In certain such embodiments, the pharmaceutical composition is suitable for administering orally. In certain other embodiments, the pharmaceutical composition is suitable for administering sublingually.

In certain embodiments of any of the foregoing methods, pharmaceutical composition is formulated as a tablet, a capsulean oil, a capsule, a gel, a paste, a powder, a suspension, a syrup, an enema, a suppository, an emulsion, a solution, an extended-release formulation, or a modified-release formulation, such as formulated as a tablet, a capsule, a paste, a powder, a suspension, a suppository, an extended-release formulation, or a modified-release formulation. In certain such embodiments, pharmaceutical composition is formulated as an extended-release formulation. In certain other embodiments, the pharmaceutical composition is formulated as a capsule.

### BRIEF DESCRIPTION OF THE DRAWINGS

FIG. 1 depicts mean (±SD) concentrations of MPZ, MPZA and MPZS following incubations of 10 μM metopimazine with pooled human liver microsomes with NADPH and no inhibitor, with NADPH in the presence of 1-ABT, a pan-cytochrome P450 inhibitor, and without NADPH or inhibitor in the incubation. MPZA is unaffected by ABT or the absence of NADPH, while MPZS is strongly inhibited by ABT and does not form in absence of NADPH.

FIG. 2 depicts Peak Area Ratio of MPZ, MPZA, MPZS and MPZH from same samples. MPZA is unaffected by CYP-inhibition or lack of NADPH, while MPZH and MPZS are inhibited in the presence of ABT and are not produced in the absence of NADPH.

FIG. 3 depicts mean (±SD) concentrations of MPZ, MPZA and MPZS following 60-min incubations of 1 μM MPZ with HLM each with specific inhibitors for seven common cytochrome P450 enzymes. MPZS formation was inhibited by the presence of the CYP3A4/5 inhibitor itraconazole.

FIG. 4 depicts the PAR of metopimazine and its metabolites in incubations of 1 μM metopimazine with specific recombinant CYP enzymes. H denotes the production of MPZH by CYP2D6, while S shows production of MPZS by CYP3A4.

FIG. 5 depicts the mean (±SD) concentrations of metopimazine, MPZA and MPZS following 60-min incubations of 1 μM metopimazine with specific recombinant CYP isoforms. MPZS was produced mainly by CYP3A4 and not CYP3A5.

FIG. 6 depicts mean (±SD) concentrations of metopimazine, MPZA and MPZS following incubations of 1 μM metopimazine in mixed, pooled HKM in triplicate in the presence of NADPH at pH 8.4. Heat-inactivation (50° C. for 5 min) and the presence and absence of the FMO inhibitor, methimazole, were all tested. No metabolites were produced under any condition.

FIG. 7 depicts mean (±SD) concentrations of MPZ, MPZA and MPZS following incubations at 1 μM MPZ with recombinant FMO enzymes.

FIG. 8 depicts formation of MPZA in nM (mean±SD) following 10 μM metopimazine incubation in HLM over 60 min in the absence and presence of 2 μM and 16 μM BNPP, a known amidase inhibitor, at pH 7.4, 8.4 and 9.4. Production of MPZA occurs at all pHs and increases at pHs above physiologic pH, including pH 9.4. The amidase inhibitor BNPP effectively inhibits formation of MPZA under all conditions.

FIG. 9 depicts the metabolism of metopimazine in humans showing the formation of the major in vivo metabolite, MPZA, formed primarily by a liver amidase and to a lesser extent by cytosolic aldehyde oxidase. Trace amounts of the CYP-mediated metabolite MPZS were also present in human samples.

FIG. 10 depicts a representative total ion chromatogram monitoring the 462.3→98.1 m/z transition to quantify MPZS. An unidentified isobaric peak was observed in some samples (marked with arrow).

### DETAILED DESCRIPTION OF THE APPLICATION

The present application provides a process of preparing metopimazine acid,

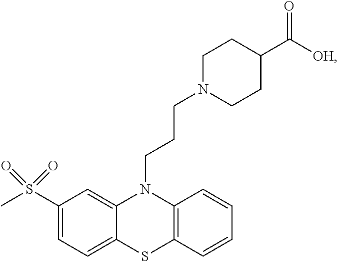 [SMILES: CS(=O)(=O)c1ccc2c(c1)N(CCCN1CCC(C(=O)O)CC1)c1ccccc1S2]

comprising contacting metopimazine,

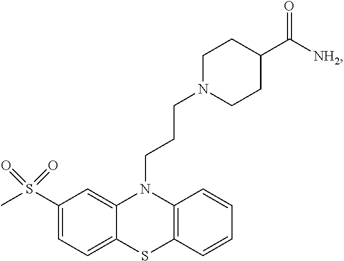 [SMILES: CS(=O)(=O)c1ccc2c(c1)N(CCCN1CCC(C(N)=O)CC1)c1ccccc1S2]

or a pharmaceutically acceptable salt thereof, with human microsomal liver amidase. The present application further provides a process of preparing metopimazine acid, comprising contacting metopimazine, or a pharmaceutically acceptable salt thereof, with human liver cytosolic aldehyde oxidase. In certain embodiments of the foregoing processes, the metopimazine, or a pharmaceutically acceptable salt thereof, is metopimazine mesylate,

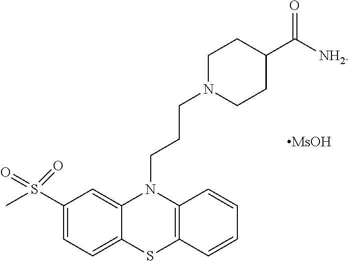 [SMILES: "·MsOH" ; CS(=O)(=O)c1ccc2c(c1)N(CCCN1CCC(C(N)=O)CC1)c1ccccc1S2]

The present application provides a process of preparing metopimazine acid comprising contacting a pharmaceutically acceptable salt of metopimazine with human microsomal liver amidase. The present application further provides a process of preparing metopimazine acid, comprising contacting a pharmaceutically acceptable salt of metopimazine with human liver cytosolic aldehyde oxidase. In certain embodiments of the foregoing processes, the pharmaceutically acceptable salt of metopimazine is metopimazine mesylate.

The present application provides a method of treating gastroparesis in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase. The present application further provides a method of treating gastroparesis in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human liver cytosolic aldehyde oxidase. In certain embodiments, the additional therapeutic agent is neither metabolized by human microsomal liver amidase nor metabolized by human liver cytosolic aldehyde oxidase. In certain embodiments of the foregoing methods of treating gastroparesis, the gastroparesis is diabetic gastroparesis. In certain other embodiments of the foregoing methods of treating gastroparesis, the gastroparesis is idiopathic gastroparesis. In certain embodiments, the gastroparesis comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the gastroparesis comprises the symptom nausea. In other such embodiments, the gastroparesis comprises the symptom vomiting. In certain embodiments of the foregoing methods of treating gastroparesis, the additional therapeutic agent is a further agent useful in the treatment of gastroparesis, such as a motilin agonist, a grelin agonist, a 5HT3 antagonist, a D2 antagonist, or a 5HT4 agonist. In other embodiments of the foregoing methods of treating gastroparesis, the additional therapeutic agent is an agent useful for the treatment of co-morbid conditions, such as an agent useful in the treatment of GERD, IBS, IBD, constipation, diarreha, or pain.

The present application provides a method of treating nausea associated with gastroparesis in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase. The present application further provides a method of treating nausea associated with gastroparesis in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human liver cytosolic aldehyde oxidase.

The present application provides a method of treating vomiting associated with gastroparesis in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase. The present application further provides a method of treating vomiting associated with gastroparesis in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human liver cytosolic aldehyde oxidase.

The present application provides a method of improving gastric emptying in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase. The present application further provides a method of improving gastric emptying in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human liver cytosolic aldehyde oxidase.

The present application provides a method of treating functional and motility disorders of the GI tract in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase. The present application further provides a method of treating functional and motility disorders of the GI tract in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human liver cytosolic aldehyde oxidase.

The present application provides a method of treating an enteric nervous system disorder in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase. The present application further provides a method of treating an enteric nervous system disorder in a human subject in need thereof, comprising administering to the subject metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human liver cytosolic aldehyde oxidase. In certain embodiments, the enteric nervous system disorder is a chronic disorder. In other embodiments, the enteric nervous system disorder is an acute disorder. In certain embodiments, the enteric nervous system disorder is selected from the group consisting of gastroparesis, Irritable Bowel Syndrome, lysosomal storage disorders, intestinal dysmotility, ganglioneuroma, multiple endocrine neoplasia type 2B (MEN2B), gastrointestinal neuropathy, functional dyspepsia, gastroesophageal reflux disease (GERD), and intestinal neuronal dysplasia. In certain embodiments, the enteric nervous system disorder comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the enteric nervous system disorder comprises the symptom nausea. In other such embodiments, the enteric nervous system disorder comprises the symptom vomiting.

In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject chronically. In certain other embodiments of any of the methods disclosed herein, the metopimazine, or a pharmaceutically acceptable salt thereof (e.g., a pharmaceutically acceptable salt of metopimazine, such as metopimazine mesylate), is administered to the subject acutely.

In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject for at least 6 days. In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject for at least 7 days. In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject for at least four weeks. In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject for at least 12 weeks.

In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject one time per day. In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject two times per day. In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject three times per day. In certain embodiments of any of the methods disclosed herein, the metopimazine, or a pharmaceutically acceptable salt thereof (e.g., a pharmaceutically acceptable salt of metopimazine, such as metopimazine mesylate), is administered to the subject four times per day. In certain embodiments of any of the methods disclosed herein, between about 5 mg and about 160 mg of the metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, more than 20 mg of metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, more than 30 mg of metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, between about 5 mg and about 240 mg of the metopimazine, or a pharmaceutically acceptable salt thereof (e.g., a pharmaceutically acceptable salt of metopimazine, such as metopimazine mesylate), is administered to the subject per day, such as about 5 mg, about 10 mg, about 15 mg, about 20 mg, about 30 mg, about 40 mg, about 50 mg, about 60 mg, about 80 mg, about 90 mg, about 100 mg, about 120 mg, about 150 mg, about 160 mg, about 180 mg, about 200 mg, about 240 mg of the metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, about 5 mg of the metopimazine, or a pharmaceutically acceptable salt thereof (e.g., a pharmaceutically acceptable salt of metopimazine, such as metopimazine mesylate), is administered to the subject one time, two times, three times, or four times per day. In certain embodiments of any of the methods disclosed herein, about 10 mg, about 20 mg, about 30 mg, about 40 mg, about 50 mg, or about 60 mg of the metopimazine, or a pharmaceutically acceptable salt thereof (e.g., a pharmaceutically acceptable salt of metopimazine, such as metopimazine mesylate), is administered to the subject one time, two times, three times, or four times per day. In certain embodiments of any of the methods disclosed herein, about 40 mg of metopimazine, or a pharmaceutically acceptable salt thereof (e.g., a pharmaceutically acceptable salt of metopimazine, such as metopimazine mesylate), is administered to the subject four times per day. In certain embodiments of any of the methods disclosed herein, about 60 mg of metopimazine, or a pharmaceutically acceptable salt thereof (e.g., a pharmaceutically acceptable salt of metopimazine, such as metopimazine mesylate), is administered to the subject four times per day.

In certain embodiments of any of the methods disclosed herein, the metopimazine, or a pharmaceutically acceptable salt thereof (e.g., a pharmaceutically acceptable salt of metopimazine, such as metopimazine mesylate), and the additional therapeutic agent are administered simultaneously in separate compositions. In certain other embodiments of any of the methods disclosed herein, the metopimazine, or a pharmaceutically acceptable salt thereof (e.g., a pharmaceutically acceptable salt of metopimazine, such as metopimazine mesylate), and the additional therapeutic agent are administered at different times and in separate compositions. In certain other embodiments of any of the methods disclosed herein, the metopimazine, or a pharmaceutically acceptable salt thereof (e.g., a pharmaceutically acceptable salt of metopimazine, such as metopimazine mesylate), and the additional therapeutic agent are administered in a composition in which both agents are present.

In certain embodiments of any of the methods disclosed herein, the metopimazine, or a pharmaceutically acceptable salt thereof (e.g., a pharmaceutically acceptable salt of metopimazine, such as metopimazine mesylate), is suitable for administering orally, intraduodenally, intracolonically, parenterally, enterally, intraperitoneally, topically, transdermally, ophthalmically, intranasally, locally, non-orally, via spray, subcutaneously, intravenously, intratonsillary, intramuscularly, buccally, sublingually, intra-arterially, intrathecally, by infusion, by inhalation, or rectally, such as orally, intraduodenally, intracolonically, enterally, topically, intranasally, non-orally, buccally, sublingually, by inhalation, or rectally. In certain such embodiments, the metopimazine, or a pharmaceutically acceptable salt thereof (e.g., a pharmaceutically acceptable salt of metopimazine, such as metopimazine mesylate), is suitable for administering orally. In certain other embodiments, the metopimazine, or a pharmaceutically acceptable salt thereof (e.g., a pharmaceutically acceptable salt of metopimazine, such as metopimazine mesylate), is suitable for administering sublingually.

In certain embodiments of any of the methods disclosed herein, the metopimazine, or a pharmaceutically acceptable salt thereof (e.g., a pharmaceutically acceptable salt of metopimazine, such as metopimazine mesylate), is formulated as a tablet, a capsulean oil, a capsule, a gel, a paste, a powder, a suspension, a syrup, an enema, a suppository, an emulsion, a solution, an extended-release formulation, or a modified-release formulation, such as formulated as a tablet, a capsule, a paste, a powder, a suspension, a suppository, an extended-release formulation, or a modified-release formulation. In certain such embodiments, the metopimazine, or a pharmaceutically acceptable salt thereof (e.g., a pharmaceutically acceptable salt of metopimazine, such as metopimazine mesylate), is formulated as an extended-release formulation. In certain other embodiments, the metopimazine, or a pharmaceutically acceptable salt thereof (e.g., a pharmaceutically acceptable salt of metopimazine, such as metopimazine mesylate), is formulated as a capsule.

In certain embodiments of any of the methods disclosed herein, the metopimazine, or a pharmaceutically acceptable salt thereof, is metopimazine mesylate.

The present application further provides a pharmaceutical composition comprising metopimazine, or a pharmaceutically acceptable salt thereof, in combination with an additional therapeutic agent, wherein the additional therapeutic agent is not metabolized by human microsomal liver amidase. The present application further provides a pharmaceutical composition comprising metopimazine, or a pharmaceutically acceptable salt thereof, in combination with an additional therapeutic agent, wherein the additional therapeutic agent is not metabolized by human liver cytosolic aldehyde oxidase. In certain embodiments, the additional therapeutic agent is neither metabolized by human microsomal liver amidase nor metabolized by human liver cytosolic aldehyde oxidase. In certain embodiments of the foregoing pharmaceutical compositions, the metopimazine, or a pharmaceutically acceptable salt thereof, is a pharmaceutically acceptable salt of metopimazine. In certain such embodiments, the pharmaceutically acceptable salt of metopimazine is metopimazine mesylate.

The present application further provides a method of treating gastroparesis in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase. In certain embodiments, the gastroparesis is diabetic gastroparesis. In other embodiments, the gastroparesis is idiopathic gastroparesis. In certain embodiments of the foregoing, the gastroparesis comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the gastroparesis comprises the symptom nausea. In other such embodiments, the gastroparesis comprises the symptom vomiting.

A method of treating nausea associated with gastroparesis in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of treating vomiting associated with gastroparesis in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of improving gastric emptying in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of treating functional and motility disorders of the GI tract in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase.

A method of treating an enteric nervous system disorder in a human subject in need thereof, comprising administering to the subject a pharmaceutical composition comprising metopimazine, or a pharmaceutically acceptable salt thereof (e.g., a pharmaceutically acceptable salt of metopimazine, such as metopimazine mesylate), in combination with an additional therapeutic agent, wherein the additional therapeutic agent is not metabolized by human microsomal liver amidase and/or not metabolized by human liver cytosolic aldehyde oxidase. In certain embodiments, the enteric nervous system disorder is a chronic disorder. In other embodiments, the enteric nervous system disorder is an acute disorder. In certain embodiments of the foregoing, the enteric nervous system disorder is selected from the group consisting of gastroparesis, Irritable Bowel Syndrome, lysosomal storage disorders, intestinal dysmotility, ganglioneuroma, multiple endocrine neoplasia type 2B (MEN2B), gastrointestinal neuropathy, functional dyspepsia, gastroesophageal reflux disease (GERD), and intestinal neuronal dysplasia. In certain embodiments, the enteric nervous system disorder comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation. In certain such embodiments, the enteric nervous system disorder comprises the symptom nausea. In other such embodiments, the enteric nervous system disorder comprises the symptom vomiting.

In certain embodiments of any of the foregoing methods, the pharmaceutical composition is administered to the subject chronically. In certain other embodiments of any of the foregoing methods, the pharmaceutical composition is administered to the subject acutely.

In certain embodiments of any of the methods disclosed herein, the pharmaceutical composition is administered to the subject for at least 6 days. In certain embodiments of any of the methods disclosed herein, the pharmaceutical composition is administered to the subject for at least 7 days. In certain embodiments of any of the methods disclosed herein, pharmaceutical composition is administered to the subject for at least four weeks. In certain embodiments of any of the methods disclosed herein, the pharmaceutical composition is administered to the subject for at least 12 weeks.

In certain embodiments of any of the methods disclosed herein, the pharmaceutical composition is administered to the subject one time per day. In certain embodiments of any of the methods disclosed herein, the pharmaceutical composition is administered to the subject two times per day. In certain embodiments of any of the methods disclosed herein, pharmaceutical composition is administered to the subject three times per day. In certain embodiments of any of the methods disclosed herein, the pharmaceutical composition is administered to the subject four times per day.

In certain embodiments of any of the methods disclosed herein, between about 5 mg and about 160 mg of the metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, more than 20 mg of metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, more than 30 mg of metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, between about 5 mg and about 240 mg of the metopimazine, or a pharmaceutically acceptable salt thereof (e.g., a pharmaceutically acceptable salt of metopimazine, such as metopimazine mesylate), is administered to the subject per day, such as about 5 mg, about 10 mg, about 15 mg, about 20 mg, about 30 mg, about 40 mg, about 50 mg, about 60 mg, about 80 mg, about 90 mg, about 100 mg, about 120 mg, about 150 mg, about 160 mg, about 180 mg, about 200 mg, about 240 mg of the metopimazine, or a pharmaceutically acceptable salt thereof (e.g., a pharmaceutically acceptable salt of metopimazine, such as metopimazine mesylate), is administered to the subject per day. In certain embodiments of any of the methods disclosed herein, about 5 mg of the metopimazine, or a pharmaceutically acceptable salt thereof (e.g., a pharmaceutically acceptable salt of metopimazine, such as metopimazine mesylate), is administered to the subject one time, two times, three times, or four times per day. In certain embodiments of any of the methods disclosed herein, about 10 mg, about 20 mg, about 30 mg, about 40 mg, about 50 mg, or about 60 mg of the metopimazine, or a pharmaceutically acceptable salt thereof (e.g., a pharmaceutically acceptable salt of metopimazine, such as metopimazine mesylate), is administered to the subject one time, two times, three times, or four times per day. In certain embodiments of any of the methods disclosed herein, about 40 mg of metopimazine, or a pharmaceutically acceptable salt thereof (e.g., a pharmaceutically acceptable salt of metopimazine, such as metopimazine mesylate), is administered to the subject four times per day. In certain embodiments of any of the methods disclosed herein, about 60 mg of metopimazine, or a pharmaceutically acceptable salt thereof (e.g., a pharmaceutically acceptable salt of metopimazine, such as metopimazine mesylate), is administered to the subject four times per day.

In certain embodiments of any of the foregoing methods, pharmaceutical composition is suitable for administering orally, intraduodenally, intracolonically, parenterally, enterally, intraperitoneally, topically, transdermally, ophthalmically, intranasally, locally, non-orally, via spray, subcutaneously, intravenously, intratonsillary, intramuscularly, buccally, sublingually, intra-arterially, intrathecally, by infusion, by inhalation, or rectally, such as orally, intraduodenally, intracolonically, enterally, topically, intranasally, non-orally, buccally, sublingually, by inhalation, or rectally. In certain such embodiments, the pharmaceutical composition is suitable for administering orally. In certain other embodiments, the pharmaceutical composition is suitable for administering sublingually.

In certain embodiments of any of the foregoing methods, pharmaceutical composition is formulated as a tablet, a capsulean oil, a capsule, a gel, a paste, a powder, a suspension, a syrup, an enema, a suppository, an emulsion, a solution, an extended-release formulation, or a modified-release formulation, such as formulated as a tablet, a capsule, a paste, a powder, a suspension, a suppository, an extended-release formulation, or a modified-release formulation. In certain such embodiments, pharmaceutical composition is formulated as an extended-release formulation. In certain other embodiments, the pharmaceutical composition is formulated as a capsule.

In certain embodiments of any of the foregoing methods, the metopimazine, or a pharmaceutically acceptable salt thereof (e.g., a pharmaceutically acceptable salt of metopimazine, such as metopimazine mesylate), is administered as a pharmaceutical composition comprising an effective amount of the metopimazine, or a pharmaceutically acceptable salt thereof (e.g., a pharmaceutically acceptable salt of metopimazine, such as metopimazine mesylate), and a pharmaceutically acceptable diluent or carrier. In certain such embodiments, the composition further comprises an additional therapeutic agent, wherein the additional therapeutic agent is not metabolized by human microsomal liver amidase. In other such embodiments, the composition further comprises an additional therapeutic agent, wherein the additional therapeutic agent is not metabolized by human liver cytosolic aldehyde oxidase.

### Definitions

As used in the specification and claims, the singular forms “a”, “an” and “the” include plural references unless the context clearly dictates otherwise. For example, the term “a cell” includes a plurality of cells, including mixtures thereof.

The term “agonist,” as used herein, generally refers to a molecule such as a compound, a drug, an enzyme activator or a hormone modulator that binds to a specific receptor and triggers a response in the cell. An agonist generally mimics the action of an endogenous ligand (such a, e.g., dopamine) that binds to the same receptor.

The term “antagonist,” as used herein, refers to a molecule such as a compound, which diminishes, inhibits, or prevents a cellular response to a receptor activated by an agonist. Antagonists can include, but are not limited to, competitive antagonists, non-competitive antagonists, uncompetitive antagonists, partial agonists and inverse agonists. Competitive antagonists can reversibly bind to receptors at the same binding site (active site) as the endogenous ligand or agonist, without necessarily activating the receptor. Non-competitive antagonists (also known as allosteric antagonists) can bind to a distinctly separate binding site from the agonist, exerting their action to that receptor via another binding site. Non-competitive antagonists generally do not compete with agonists for binding. Binding of a non-competitive antagonist to the receptor may result in a decreased affinity of an agonist to that receptor. Alternatively, binding of a non-competitive antagonist to a receptor may prevent a conformational change in the receptor required for agonist-mediated receptor activation. Uncompetitive antagonists may require receptor activation by an agonist before they can bind to a separate allosteric binding site. Partial agonists can refer to molecules which, at a given receptor, might differ in the amplitude of the functional response that they elicit after maximal receptor occupancy. Although they are agonists, partial agonists can act as a competitive antagonist if co-administered with a full agonist, as it competes with the full agonist for receptor occupancy and producing a net decrease in the receptor activation observed with the full agonist alone. An inverse agonist can have effects similar to an antagonist, but causes a distinct set of downstream biological responses. Constitutively active receptors which exhibit intrinsic or basal activity can have inverse agonists, which not only block the effects of binding agonists like a classical antagonist, but also inhibit the basal activity of the receptor.

As used herein, “gastrointestinal (GI) tract” refers to portions of the digestive tract where substantial absorption is observed. As one of skill would readily appreciate, substantial absorption is generally observed in the oral cavity, small intestine (e.g., duodenum, jejunum, and ileum), and large intestine (e.g., colon).

As used herein, “metopimazine mesylate” refers to 1-(3-(2-(methylsulfonyl)-10H-phenothiazin-10-yl)propyl)piperidine-4-carboxamide methanesulfonic acid.

As used herein, the “oral cavity” generally refers to the mouth and includes the lips, the lining inside the cheeks and lips, the tongue, the upper and lower gums, the floor of the mouth under the tongue, the sublingual mucosa, the roof of the mouth, and the area behind the wisdom teeth.

As used herein, a compound that is “peripherally restricted” generally refers to a compound that does not substantially cross an intact blood brain barrier of a subject. The term also encompasses compounds that may cross an intact blood brain barrier, but upon administration to a subject is rapidly metabolized to a form that does not substantially cross an intact blood brain barrier of the subject. A compound may be considered “peripherally restricted” if, upon administration to a subject, less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1% of the compound crosses an intact blood brain barrier of the subject.

The term “solvate” refers to crystalline solid adducts containing either stoichiometric or nonstoichiometric amounts of a solvent incorporated within the crystal structure. Therefore, the term “non-solvate” form herein refers to salt crystals that are free or substantially free of solvent molecules within the crystal structures of the invention. Similarly, the term “non-hydrate form herein refers to salt crystals that are free or substantially free of water molecules within the crystal structures of the invention.

As used herein, the terms “treatment” or “treating” are used interchangeably herein. These terms refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying or eliminating the onset of symptoms of a disease or condition, slowing, halting, or reversing the progression of a disease or condition, or any combination thereof. For prophylactic benefit, the compositions may be administered to a subject at risk of developing a particular disease, or to a subject reporting one or more of the physiological symptoms of a disease, even though a diagnosis of this disease may not have been made.

A “sub-therapeutic amount” of an agent is an amount less than the effective amount for that agent. When combined with an effective or sub-therapeutic amount of one or more additional agents, the sub-therapeutic amount can produce a result desired by the physician, due to, for example, synergy in the resulting efficacious effects, or reduced adverse effects.

A “synergistically effective” therapeutic amount or “synergistically effective” amount of an agent or therapy is an amount which, when combined with an effective or sub-therapeutic amount of one or more additional agents, produces a greater effect than when either of the agents are used alone. In some embodiments, a synergistically effective therapeutic amount of an agent or therapy produces a greater effect when used in combination than the additive effects of any of the individual agents when used alone. The term “greater effect” encompasses not only a reduction in symptoms of the disorder to be treated, but also an improved side effect profile, improved tolerability, improved patient compliance, improved efficacy, or any other improved clinical outcome.

The term “co-administration,” “administered in combination with,” and their grammatical equivalents, as used herein, encompass administration of two or more agents to an animal so that both agents and/or their metabolites are present in the subject at the same time. Co-administration includes simultaneous administration in separate compositions, administration at different times in separate compositions, or administration in a composition in which both agents are present.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

Exemplary Subjects

The pharmaceutical compositions and/or combinations as disclosed herein can be used for the treatment of a disorder in a subject in need thereof. The subject may be suffering from, may be diagnosed with, may be exhibiting a symptom of, or may be suspected of having the disorder. The disorder can be a gastrointestinal disorder, an enteric nervous system disorder, or other disorder. The disorder may be characterized by a hypomotility of at least a portion of the gastrointestinal tract. For example, the disorder can be characterized by hypomotility of the stomach and/or intestine. The hypomotility may be caused by aberrant ENS neuronal signaling, for example, by aberrant dopamine signaling activity.

In some embodiments, the enteric nervous system disorder is gastroparesis. The terms “gastroparesis” and “delayed gastric emptying” are used interchangeably herein to refer to a disorder that, e.g., slows or stops the movement of food from the stomach to the small intestine. Normally, the muscles of the stomach, which are controlled by the vagus nerve, contract to break up food and move it through the gastrointestinal (GI) tract. Gastroparesis can occur, for example, when the vagus nerve is damaged by illness or injury, causing the stomach muscles stop working normally. In subjects with gastroparesis, food can move slowly from the stomach to the small intestine or may stop moving altogether. Accordingly, the subject may be suffering from, may be diagnosed with, may be exhibiting a symptom of, or may be suspected of having gastroparesis.

A subject may be suspected of having gastroparesis if the subject exhibits or has exhibited a symptom of gastroparesis. Symptoms of gastroparesis can include gastroesophageal reflux (GER), also called acid reflux or acid regurgitation. Gastroesophageal reflux generally refers to a condition in which stomach contents flow back up into the esophagus. Other symptoms associated with gastroparesis include, but are not limited to, early satiety, postprandial fullness, abdominal fullness, abdominal pain and/or burning sensation in the stomach area, abdominal bloating, lack of appetite, anorexia, malnutrition, nausea, and vomiting. A symptom of gastroparesis can be mild, moderate or severe, and can occur frequently or infrequently. A symptom of gastroparesis can vary in severity over time in the same subject. Accordingly, the subject may exhibit or has exhibited GER, early satiety, postprandial fullness, abdominal fullness, abdominal pain and/or burning sensation in the stomach area, abdominal bloating, lack of appetite, anorexia, malnutrition, nausea, and/or vomiting.

The subject may be diagnosed with gastroparesis. Gastroparesis may be diagnosed by any means known to those of skill in the art or otherwise described herein. Gastroparesis may be diagnosed, e.g., through a physical exam, medical history, blood tests, tests to rule out blockage or structural problems in the GI tract, gastric emptying assays, and assays of GI contractile activity. Tests may also identify a nutritional disorder or underlying disease. Tests that are useful in diagnosing gastroparesis include, but are not limited to, upper gastrointestinal (GI) endoscopy, upper GI series, ultrasound tests, gastric emptying scintigraphy, gastric emptying breath test, antral manometry, electrogastrography, and/or electrogastroenterography.

Upper GI endoscopy can be used to rule out other conditions that could result in delayed gastric emptying (such as, e.g., a physical obstruction). Upper GI endoscopy typically involves use of an endoscope (e.g., a small, flexible tube with a light) to visualize the upper GI tract, including, e.g., the esophagus, stomach, and duodenum (the first part of the small intestine). The endoscope is generally used to image the stomach and/or duodenum. A small camera mounted on the endoscope can transmit a video image to a monitor, allowing close examination of the intestinal lining. Upper GI endoscopy may show physical blockage of the upper GI tract, for example, a large bezoar (e.g., solid collections of food, mucus, vegetable fiber, hair, or other material). In some embodiments, the subject is diagnosed with gastroparesis if the subject exhibits a symptom of gastroparesis and upper GI endoscopy does not reveal a physical blockage causing the delayed gastric emptying.

An upper GI series may be performed to look at the small intestine. The test may be performed at a hospital or outpatient center by an x-ray technician, and the images may be interpreted by a radiologist. During the procedure, the subject may stand or sit in front of an x-ray machine and drink barium, a chalky liquid. Barium may coat the small intestine, making signs of gastroparesis show up more clearly on x rays. Gastroparesis may be indicated in cases wherein the x-ray shows food in the stomach after fasting. In some embodiments, the subject is diagnosed with gastroparesis if an upper GI series reveals food in the stomach after fasting.

Ultrasound can be useful in ruling out other syndromes which may share symptoms in common with gastroparesis. Such other syndromes include gallbladder disease and pancreatitis. Ultrasound generally uses a device, called a transducer, that bounces safe, painless sound waves off organs to create an image of their structure. The procedure can be performed in a health care provider's office, outpatient center, or hospital by a specially trained technician. Ultrasound images may be interpreted by a radiologist. The subject may be diagnosed with gastroparesis if the subject exhibits a symptom of gastroparesis and other syndromes such as, e.g., gallbladder disease, pancreatitis, are ruled out by, for example, ultrasound.

Gastric emptying scintigraphy can be used to diagnose gastroparesis in a subject. Gastric emptying scintigraphy can involve ingestion of a bland meal—such as eggs or an egg substitute—that contains a small amount of radioactive material. The radioactive material may be 99-M Technetium (TC) sulfur colloid or other radioactive ligand. The test may be performed in a radiology center or hospital. An external camera may be used to detect and/or measure radioactivity in the abdominal region. Radioactivity may be measured at timed intervals, e.g., at 1, 2, 3, and 4 hours after the meal. Gastroparesis may be positively identified in subjects exhibiting more than 10 percent of the meal within the stomach at 4 hours. Other measures of gastric emptying include, but are not limited to, the time at which 50% of the meal has been emptied out of the stomach. See, e.g., Thomforde, G. M. et al., Evaluation of an inexpensive screening scintigraphic test of gastric emptying, 36 J. Nucl. Med. 93 (1995), hereby incorporated by reference. In some embodiments, the subject is diagnosed with gastroparesis via gastric emptying scintigraphy.

A breath test useful for assessing gastric emptying can utilize radioactively labeled food (e.g., labeled with C¹³-octanoic acid). C¹³ from the food may be absorbed when it reaches the small bowel. The absorbed C¹³ can then be rapidly metabolized in the liver to produce 13 CO₂. The produced 13 CO₂ may then be detected in the breath of the subject. The subject's breath may be collected and sampled at defined intervals. The samples may be analyzed for 13 CO₂ by any means known in the art. The rate of appearance of 13 CO₂ in the breath can be used to indicate the rate of gastric emptying. An exemplary method of performing a C¹³-octanoic acid breath test is described in Ghoos, Y. S., et al., 104 Gastroenterology 1640-1647 (1993), hereby incorporated by reference. In some embodiments, the subject is diagnosed with gastroparesis via a breath test.

Manometry generally refers to the assessment of pressure changes in a lumen. Antral manometry, which can also be referred to as antro-duodenal manometry, generally refers to techniques for the evaluation of contractile activity in the distal stomach and duodenum. Intraluminal pressure of the stomach and/or duodenum can be measured through pressure sensors which are introduced into the lumen via a catheter. Measurements may be recorded over time in order to assess intraluminal pressure changes. Recordings may last for any amount of time. Intraluminal pressure changes can be used to indicate contractile patterns in the stomach and/or duodenum. Intraluminal pressure changes may be measured in a fasting state and/or after ingestion of a meal (post-prandially). Post-prandial contractile hypomotility can be indicative of gastroparesis in a subject. Accordingly, a subject may exhibit post-prandial gastric hypomotility, as determined by manometry.

Electrogastrography generally refers to techniques and methods for recording electrical activity of the stomach. Likewise, electrogastroenterography refers to techniques and methods for recording electrical activity of the stomach and small intestine. Such electrical activity can be recorded from the gastrointestinal mucosa, serosa, or the outer skin surface (cutaneously). Gastrointestinal mucosa can refer to the mucous membrane layer of the GI tract. Gastrointestinal serosa can comprise a thin layer of cells which secrete serous fluid, and a thin epithelial layer. Recordings can be made during a fasting state, and after ingestion of a meal (usually 60 minutes). Deviations from the normal frequency of electrical activity can include bradygastria and/or tachygastria. Control subjects typically exhibit an increase in electrical activity after a meal, indicative of increased GI motility. Subjects with aberrant GI motility can exhibit abnormal rhythms in activity and/or impairments in the postprandial increase. A normal frequency of GI electrical activity can be, e.g., 3 cycles per minute. Bradygastria, which can be characterized as a frequency of GI electrical activity that is decreased from normal, e.g., that is less than 2 cycles per minute for at least one minute, can be indicative of gastroparesis. In some embodiments, a subject may exhibit bradygastria. Electrogastrography (EGG) which measures electrical activity with cutaneous electrodes similar to those used in electrocardiograms can also be used to diagnose gastroparesis. (Stern, R. N. et al. EGG: Common issues in validation and methodology, 24 Psychophysiology 55-64 (1987)), hereby incorporated by reference. Accordingly, a subject may be diagnosed with gastroparesis as determined by electrogastrography.

The subject may be suffering from, may be diagnosed with, may be exhibiting a symptom of, or may be suspected of having gastroesophageal reflux disease (GERD). GERD can be a chronic condition resulting in gastroesophageal reflux. Symptoms of GERD include, e.g., heartburn, dry, chronic cough, wheezing, asthma, recurrent pneumonia, nausea, vomiting, sore throat, difficulty swallowing, pain in the chest or upper abdomen, dental erosion, bad breath, spitting up. GERD may be diagnosed with the aid of tests. Tests that are useful in the diagnosis of GERD include, e.g., upper GI series, described herein, upper endoscopy, esophageal pH monitoring, and esophageal manometry.

The subject may be suffering from, may be diagnosed with, may be exhibiting a symptom of, or may be suspected of having enteric nervous system disorder which is associated with a vestibular disorder of the ear. The vestibular disorder of the ear can be Menetrier's disease. Menetrier's disease can be characterized by enlargement of ridges (also referred to herein as rugae) along the inside of the stomach wall, forming giant folds in the lining of the stomach. Ménétrier disease may also cause a decrease in stomach acid resulting from a reduction in acid-producing parietal cells. Symptoms of Ménétrier disease include, by way of example only, severe stomach pain, nausea, frequent vomiting, and the like.

The subject may be suffering from, may be diagnosed with, may be exhibiting a symptom of, or may be suspected of having cyclical vomiting syndrome (CVS). Cyclical vomiting syndrome can be characterized by episodes or cycles of severe nausea and vomiting that alternate with symptom-free intervals. Such episodes can last for hours, or even days. Episodes can start at the same time of day, can last the same length of time, and can occur with the same symptoms and level of intensity. Episodes can be so severe that a person has to stay in bed for days, unable to go to school or work. Other symptoms of cyclical vomiting syndrome include, e.g., abdominal pain, diarrhea, fever, dizziness, and sensitivity to light during vomiting episodes. Continued vomiting may cause severe dehydration that can be life threatening. Symptoms of dehydration include thirst, decreased. Cyclical vomiting syndrome may be diagnosed in a subject who has experienced the following symptoms for at least 3 months: vomiting episodes that start with severe vomiting—several times per hour—and last less than 1 week, three or more separate episodes of vomiting in the past year, and absence of nausea or vomiting between episodes.

The subject may be suffering from, may be diagnosed with, may be exhibiting a symptom of, or may be suspected of having Irritable Bowel Syndrome (IBS). IBS generally refers to a syndrome with multiple subtypes in which subjects experience recurrent or chronic gastrointestinal symptoms. Symptoms of IBS can include, e.g., abdominal pain, abdominal discomfort, constipation, diarrhea, mucus in the stool, abdominal bloating, or a combination of any of the above. IBS may be diagnosed when a person has had abdominal pain or discomfort at least three times a month for the last 3 months without other disease or injury that could explain the pain. The pain or discomfort of IBS may occur with a change in stool frequency or consistency or be relieved by a bowel movement. IBS can be classified into four subtypes based on a subject's usual stool consistency. The four subtypes of IBS are: IBS with constipation (IBS-C), IBS with diarrhea (IBS-D), mixed IBS (IBS-M), and unsubtyped IBS (IBS-U). A subject with IBS-C may have hard or lumpy stools at least 25 percent of the time, may have loose or watery stools less than 25 percent of the time, or a combination of the two. A subject with IBS-D may have loose or watery stools at least 25 percent of the time, hard or lumpy stools less than 25 percent of the time, or a combination of the two. A subject with IBS-M may have hard or lumpy stools at least 25 percent of the time and loose or watery stools at least 25 percent of the time. A subject with IBS-U may have hard or lumpy stools less than 25 percent of the time, loose or watery stools less than 25 percent of the time, or a combination of the two. Constipation associated with IBS may be due to slow or delayed gastric motility. In some embodiments, the subject with IBS has experienced constipation. IBS can be diagnosed in a subject by any means known in the art or otherwise described herein. For instance, IBS may be diagnosed by a health care provider. The health care provider may conduct a physical exam and may take a medical history of the subject. IBS may be diagnosed if a subject has exhibited one or more symptoms of IBS for at least 3, 4, 5, or 6 months, with one or more symptoms occurring at least three times a month for the previous 3 months. Additional tests that may be useful in the diagnosis of IBS include, but are not limited to: a stool test, lower GI series, flexible sigmoidoscopy, or colonoscopy.

The subject may be suffering from, may be diagnosed with, may be exhibiting a symptom of, or may be suspected of having functional dyspepsia (e.g., impaired digestion). Symptoms of dyspepsia include but are not limited to, e.g., chronic or recurrent pain in the upper abdomen, upper abdominal fullness, postprandial fullness, early satiety, bloating, belching, nausea, vomiting, heartburn, sour taste in the mouth. Functional dyspepsia (e.g., nonulcer dyspepsia) generally refers to dyspepsia without evidence of an organic disease that is likely to explain the symptoms of dyspepsia. An example of functional dyspepsia is dyspepsia in the absence of an ulcer. Functional dyspepsia is estimated to affect about 15% of the general population in western countries. Other exemplary ENS disorders include but are not limited to, e.g., intestinal dysmotility, ganglioneruoma, multiple endocrine neoplasia type 2B (MEN2B), gastrointestinal neuropathy, and intestinal neuronal dysplasia.

The subject may be suffering from, may be diagnosed with, may be exhibiting a symptom of, or may be suspected of having an enteric nervous system disorder caused by another underlying disease. For example, the enteric nervous system disorder can be a Parkinson's disease-induced ENS disorder. Parkinson's disease-induced ENS disorder can be related to degeneration of dopamine ENS neurons. Symptoms of a Parkinson's disease-induced ENS disorder include, e.g., constipation, nausea, vomiting, and the like. In some embodiments, a subject to be treated according to a method of the application is diagnosed with, suffering a symptom of, is suspected of having, Parkinson's disease, and further exhibits a symptom of an ENS disorder as described herein.

The subject may be suffering from, may be diagnosed with, may be exhibiting a symptom of, or may be suspected of having an enteric nervous system disorder can associated with scleroderma. Scleroderma can be characterized by hardening and tightening of the skin and connective tissues. In some embodiments, the subject is suffering from, may be diagnosed with, may be exhibiting a symptom of, or may be suspected of having gastroparesis associated with Scleroderma

The subject may be suffering from, may be diagnosed with, may be exhibiting a symptom of, or may be suspected of having a diabetes-associated enteric nervous system disorder. The diabetes-associated enteric nervous system disorder can be a diabetes-associated gastroparesis. The subject may be suffering from, may be diagnosed with, may be exhibiting a symptom of, or may be suspected of having an enteric nervous system disorder associated with multiple sclerosis.

Other diseases and clinical conditions that can cause an enteric nervous system disorder such as gastroparesis include, e.g., cancer, hypothyroidism, hyperthyroidism, hyperparathyroidism, adrenal insufficiency (Addison's disease), gastric ulcer, gastritis, post-gastric surgery, such as, e.g., vagotomy (resection of the vagus nerve), antrectomy (resection of a portion of the stomach distal to the antrum of the stomach), subtotal gastrectomy (resection of a gastric tumor), gastrojejunostomy (a surgical procedure that connects two lumens of the GI tract, such as a proximal segment of stomach and a segment of the small intestine), fundoplication (a surgical procedure that wraps an upper portion of the stomach around a lower end of the esophagus), polymyositis (a persistent inflammatory muscle disease that can cause muscle weakness), muscular dystrophy (a disease that can cause progressive muscle weakness), amyloidosis (characterized by buildup of amyloid in a tissue or organ of the subject, such as in the gastrointestinal tract), intestinal pseudo-obstruction (a condition that causes symptoms that are associated with bowel obstruction but wherein no bowel obstruction is found), dermatomyositis (a disease characterized by muscular inflammation), systemic lupus erythematosus (a systemic autoimmune disease that can affect various tissues of the body, including the nervous system), eating disorders such as, e.g., anorexia and bulimia, depression, paraneoplastic syndrome, and high cervical cord lesions (e.g., lesions at spinal cord C4 or above).

The subject can be suffering a symptom of an enteric nervous system disorder. Exemplary symptoms are described herein. In some embodiments, the symptom is nausea and/or vomiting. In some embodiments, the cause of the symptom is unknown (e.g., unexplained nausea). In some embodiments, the symptom is a chronic or recurrent symptom. The subject may, for example, experience the symptom for at least 3 days, at least 5 days, at least 1 week, at least 2 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 1.5 years, at least 2 years, at least 3 years, at least 4 years, at least 5 years, at least 6 years, at least 7 years, at least 8 years, at least 9 years, or at least 10 years. The subject may experience the symptom 1, 2, 3, 4, 5, 6, 7, 8, 8, 10, 11, 12, 13, 14, 15, 16, 17, 18, 19, 20, 21, 22, 23, 24, 25, 26, 27, 28, 29, 30, 31, or more than 31 times a month.

The subject may be, e.g., a mouse, a rat, a hamster, a gerbil, a dog, a cat, a primates such as, e.g., a monkey or human. In some embodiments, the subject is a human. The subject may be an adult, a child, or an infant. The subject can be of any age.

Use of the Pharmaceutical Compositions and/or Combinations

Pharmaceutical compositions and/or combinations as described herein can be safely administered to a subject. Pharmaceutical compositions and/or combinations as described herein can be administered without necessarily increasing risk of developing a deleterious cardiac side effect. For example, pharmaceutical compositions and/or combinations described herein may not increase risk of modulating cardiac action potential, and/or may not increase risk of inducing long QT syndrome, and/or may not increase risk of cardiac arrest, and/or may not increases risk of sudden death by cardiac arrest.

The subject may be safely administered an effective amount of a pharmaceutical composition and/or combination as described herein for an unlimited amount of time. The subject may be safely administered an effective amount of the pharmaceutical composition and/or combination acutely or chronically. For example, the subject may be safely administered an effective amount of the pharmaceutical composition and/or combination once, for one day, for at least 2 days, for at least 3 days, for at least four days, for five days, for at least five days, for at least six days, for at least seven days (1 week), for at least 2 weeks, for at least 3 weeks, for at least 4 weeks, for at least 5 weeks, for at least 6 weeks, for at least 7 weeks, for at least 8 weeks, for at least 9 weeks, for at least 10 weeks, for at least 11 weeks, for at least 12 weeks, for at least 3 months, for at least 4 months, for at least 5 months, for at least 6 months, for at least 7 months, for at least 8 months, for at least 9 months, for at least 10 months, for at least 11 months, for at least 12 months (1 year), for at least 2 years, for at least 5 years, or for at least a decade.

Administration of a pharmaceutical composition and/or combination as described herein may confer an acceptable risk that the subject will develop an unwanted cardiac side effect. Risk of pharmaceutical composition and/or combination administration on developing such unwanted cardiac side effect can be determined by any means known in the art, or as described herein. For example, risk can be determined by comparing the incidence of sudden death in a population of subjects administered the pharmaceutical composition and/or combination as compared to incidence of sudden death in a population of control subjects that have not been administered the pharmaceutical composition and/or combination. Risk can be determined by tracking the number of subjects administered the pharmaceutical composition and/or combination who experienced the unwanted cardiac side effect, and the number of subjects administered the pharmaceutical composition and/or combination who did not experience the unwanted cardiac side effect. For example, if a=the number of subjects administered the pharmaceutical composition and/or combination and/or combination who experienced the unwanted cardiac side effect, and b=the number of subjects administered the pharmaceutical composition and/or combination who did not experience the unwanted cardiac side effect, the risk of experiencing the unwanted cardiac side effect conferred by being administered the pharmaceutical composition and/or combination can be calculated as a/(a+b). Relative risk (RR) may be used to compare the risk of developing an unwanted cardiac side effect conferred by administration of the pharmaceutical composition and/or combination to the risk of developing the unwanted cardiac side effect in a population of subjects that have not been administered the pharmaceutical composition and/or combination. For example, if a=the number of subjects administered the pharmaceutical composition and/or combination who experienced the unwanted cardiac side effect, b=the number of subjects administered the pharmaceutical composition and/or combination who did not experience the unwanted cardiac side effect, c=the number of subjects not administered the pharmaceutical composition and/or combination who experienced the unwanted cardiac side effect, and d=the number of subjects not administered the pharmaceutical composition and/or combination who did not experience the unwanted cardiac side effect, RR conferred by administration of the pharmaceutical composition and/or combination can be calculated as a/(a+b)/(c/(c+d). For other example, risk can be determined by calculating an odds ratio.

The RR of administration of a pharmaceutical composition and/or combination as described herein with sudden cardiac death can be less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.05, about 1, or less than 1.

The odds ratio of administration of a pharmaceutical composition and/or combination described herein with sudden cardiac death can be an acceptable odds ratio. The term odds ratio (OR) generally refers to a measure of association between an exposure (e.g., exposure to a drug) and an outcome (e.g., sudden cardiac death). The OR can represent the odds that the outcome will occur given a particular exposure, as compared to the odds of the outcome occurring in the absence of that exposure. Odds ratios can be used in case-control studies, as well as in cross-sectional and cohort study design studies. For example, if a=the number of subjects administered the pharmaceutical composition and/or combination who experienced the unwanted cardiac side effect, b=the number of subjects administered the pharmaceutical composition and/or combination who did not experience the unwanted cardiac side effect, c=the number of subjects not administered the pharmaceutical composition and/or combination who experienced the unwanted cardiac side effect, and d=the number of subjects not administered the pharmaceutical composition and/or combination who did not experience the unwanted cardiac side effect, OR conferred by administration of the pharmaceutical composition and/or combination can be calculated as ad/bc.

The OR of administration of a pharmaceutical composition and/or combination described herein with sudden cardiac death can be less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.05, about 1, or less than 1.

Unlike other dopamine modulating drugs previously indicated for the treatment of ENS, the pharmaceutical compositions and/or combinations described herein for use in the treatment of ENS are peripherally restricted. Accordingly, such pharmaceutical compositions and/or combinations can be safely administered to a subject without increasing risk in the subject for developing motor-related dysfunction mediated by brain dopaminergic signaling. For example, such pharmaceutical compositions and/or combinations can be safely administered to a subject without increasing risk in the subject for developing an extrapyramidal side effect. Exemplary extrapyramidal side effects include, e.g., tardive dyskinesia (involuntary asymmetrical movements of the muscles), dystonia (characterized by sustained muscle contractions), akinesia (lack of movement), akathisia (feeling of motor restlessness), bradykinesia (slowed movements), stiffness, and tremor, twisting and/or repetitive movements, abnormal postures, muscle spasms, e.g., muscle spasms of the neck (torticollis), muscle spasms of the eyes (oculogyric crisis) tongue spasms, spasms of the jaw, and the like. Extrapyramidal symptoms can be assessed by any means known in the art or otherwise described herein. For example, extrapyramidal symptoms may be assessed using the Simpson-Angus Scale (SAS) and/or the Barnes Akathisia Rating Scale (BARS). In some embodiments the odds ratio of administration of the pharmaceutical compositions and/or combinations described herein for use in treating an enteric nervous system disorder with incidence of an extrapyramidal side effect is less than 4, less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.05, about 1, or less than 1.

The pharmaceutical compositions and/or combinations of the application can promote gastric motility upon administration to the subject. Such pharmaceutical compositions and/or combinations may promote gastric motility by, for example, reducing dopamine D2-receptor mediated signaling in an enteric neuron of the subject. For example, the pharmaceutical compositions and/or combinations can antagonize dopamine D2 receptors in an enteric neuron of the subject. For other example, the pharmaceutical compositions and/or combinations may reduce the dopaminergic neurotransmission of an enteric neuron.

Gastric motility can be assessed by any means known to those of skill in the art or otherwise described herein. For example, gastric motility can be assessed by antral manometry, or by methods useful in the diagnosis of gastroparesis. Exemplary methods useful in the diagnosis of gastroparesis are described herein.

Administration of the pharmaceutical compositions and/or combinations as described herein can improve gastric motility as compared to a control subject and/or control population. The control subject can be an individual that has not been administered a pharmaceutical composition and/or combination described herein. A control population can be a plurality of individuals that have not been administered a pharmaceutical composition and/or combination described herein. The control subject can be a subject that is suffering from, that has been diagnosed with, be suspected of having, or exhibiting a symptom of an ENS disorder, that is not administered a pharmaceutical composition and/or combination as described herein. The control subject does not necessarily need to be a different individual, but may be the same subject at a time point prior to receiving a dose of a pharmaceutical composition and/or combination as described herein. The control subject may be the same subject at a time point subsequent to receiving a dose of a pharmaceutical composition and/or combination as described herein, after a sufficient time has passed such that the pharmaceutical composition and/or combination is no longer acting in the subject. The control subject can be a different subject. In some embodiments, administration of the pharmaceutical composition and/or combination increases gastric motility by at least 25%, 30%, 35%, 40%, 45%, 50%, 55%, 60%, 65%, 70%, 75%, 80%, 85%, 90%, 95%, 100%, or over 100% as compared to a control subject.

In some embodiments, administration of a pharmaceutical composition and/or combination described herein is effective in treating a symptom of an enteric nervous system disorder in the subject. Exemplary symptoms are described herein. The symptom may be selected from the group consisting of nausea, vomiting, delayed gastric emptying, diarrhea, abdominal pain, gas, bloating, gastroesophageal reflux, reduced appetite, weight loss, and constipation. In particular cases, administration of a pharmaceutical composition and/or combination described herein reduces nausea in the subject. Administration of a pharmaceutical composition and/or combination as described herein may reduce severity of any of the symptoms described herein. In some cases, administration of a pharmaceutical composition and/or combination as described herein reduces symptom severity by 1-5%, 2-10%, 5-20%, 10-30%, 20-50%, 40-70%, 50-80%, 70-90%, 80-95%, 90-100%. In some cases, administration of a pharmaceutical composition and/or combination as described herein reduces symptom severity by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, or more than 90%.

Administration of a pharmaceutical composition and/or combination as described herein may reduce frequency of onset of a symptom. In some cases, administration of a pharmaceutical composition and/or combination as described herein reduces frequency of symptom onset by 1-5%, 2-10%, 5-20%, 10-30%, 20-50%, 40-70%, 50-80%, 70-90%, 80-95%, 90-100%. In some cases, administration of a pharmaceutical composition and/or combination as described herein reduces frequency of symptom onset by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, or more than 90%. In some cases, administration of a pharmaceutical composition and/or combination as described herein reduces frequency of symptom onset to less than 1 episode a day, less than 1 episode a week, less than 2 episodes a month, less than 1 episode a month, less than 1 episode every 2 months, less than 1 episode every 3 months, less than 1 episode every 4 months, less than 1 episode every 5 months, less than 1 episode every 6 months, less than 1 episode every 7 months, less than 1 episode every 8 months, less than 1 episode every 9 months, less than 1 episode every 10 months, less than 1 episode every 11 months, or less than 1 episode every 12 months (1 year).

hERG channel inhibition can be determined by any means known in the art or otherwise described herein. hERG channel inhibition can be assessed in vitro, for example, by utilizing hERG expressing cultured cells. hERG-expressing cultured cells for the purposes of assessing hERG channel inhibition are available from a number of commercial vendors, such as, .e.g., Life Technologies, Cyprotex, and the like. hERG channel inhibition can be assessed by a variety of means known in the art, including, e.g., voltage clamp studies, hERG binding assays, and the like. Voltage clamp studies can employ the use of commercially available high throughput systems. Exemplary high-throughput systems are described in, e.g., U.S. Pat. No. 8,329,009, and US Patent Application Pub. No. 20020164777, which are hereby incorporated by reference. hERG binding assays can include competition and/or saturation binding assays using 3H dofetilide. Such assays are described in J Pharmacol Toxicol Methods. 2004 November-December; 50(3):187-99, which is hereby incorporated by reference. hERG channel inhibition can be determined by in vivo studies, for example, by assessment of cardiac action potentials in large animal models, e.g., canines.

Minimal hERG inhibition can be evidenced by an IC₅₀ that is higher than 0.1 μM, higher than 0.2 μM, higher than 0.3 μM, higher than 0.4 μM, higher than 0.5 μM, higher than 0.6 μM, higher than 0.7 μM, higher than 0.8 μM, higher than 0.9 μM, higher than 1 μM, higher than 2 μM, higher than 3 μM, higher than 4 μM, higher than 5 μM, higher than 6 μM, higher than 7 μM, higher than 8 μM, higher than 9 μM, higher than 10 μM, higher than 15 μM, higher than 20 μM, higher than 30 μM, higher than 40 μM, higher than 50 μM, higher than 60 μM, higher than 70 μM, higher than 80 μM, higher than 90 μM, or higher than 100 μM.

Minimal hERG inhibition can also be evidenced by measuring, at any given dose of a drug, the % inhibition of hERG-mediated tail current. hERG-mediated tail current can be measured by voltage clamp studies, e.g., by patch clamps studies. For example, hERG-mediated tail current can be measured in an hERG-expressing cell prior to contact of the cell with a test agent. hERG-mediated tail current can then be measured in the hERG-expressing cell after contact with a dose of the test agent. The differences between the hERG-mediated tail current before and after administration of the test agent can be used to determine the extent to which the test agent inhibited hERG-mediated tail current. A suitable agent for use in the disclosed methods can, at a 1 μM dose, inhibit hERG-mediated tail current by less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.4%, less than 0.3%, less than 0.25%, less than 0.2%, less than 0.15%, or less than 0.1%. A suitable agent for use in the disclosed methods can, at a 100 nM dose, inhibit hERG-mediated tail current by less than 20%, less than 15%, less than 10%, less than 9%, less than 8%, less than 7%, less than 6%, less than 5%, less than 4%, less than 3%, less than 2%, less than 1%, less than 0.5%, less than 0.4%, less than 0.3%, less than 0.25%, less than 0.2%, less than 0.15%, or less than 0.1%. In some embodiments, metopimazine can, at a 3 μM dose, inhibit hERG-mediated tail current by less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.4%, less than 0.3%, less than 0.25%, less than 0.2%, less than 0.15%, or less than 0.1%. In some embodiments, metopimazine acid can, at a 10 μM dose or higher, inhibit hERG-mediated tail current by less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.4%, less than 0.3%, less than 0.25%, less than 0.2%, less than 0.15%, or less than 0.1%.

Exemplary Compounds

It should be understood that a reference to metopimazine, or a pharmaceutically acceptable salt thereof (e.g., a pharmaceutically acceptable salt of metopimazine, such as metopimazine mesylate), includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

In certain embodiments, the metopimazine, or a pharmaceutically acceptable salt thereof, is anhydrous metopimazine mesylate. In certain embodiments, the metopimazine, or a pharmaceutically acceptable salt thereof, is metopimazine mesylate hydrate. In certain such embodiments, the metopimazine mesylate hydrate is metopimazine mesylate monohydrate. In certain embodiments, metopimazine mesylate as described herein includes a mixture of anhydrous metopimazine mesylate and metopimazine mesylate hydrate. In certain such embodiments, the mixture includes between about 10% metopimazine mesylate hydrate and about 100% metopimazine mesylate hydrate. For example, the mixture includes about 10% metopimazine mesylate hydrate, about 20% metopimazine mesylate hydrate, about 30% metopimazine mesylate hydrate, about 40% metopimazine mesylate hydrate, about 50% metopimazine mesylate hydrate, about 60% metopimazine mesylate hydrate, about 70% metopimazine mesylate hydrate, about 80% metopimazine mesylate hydrate, about 90% metopimazine mesylate hydrate, or about 95% metopimazine mesylate hydrate.

Metopimazine mesylate as described herein may be in various forms, including but not limited to, amorphous forms, milled forms and nano-particulate forms. In addition, metopimazine mesylate as described herein include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Exemplary Pharmaceutical Compositions

Pharmaceutical compositions utilized in the methods of the application may include a pharmaceutically acceptable carrier. The pharmaceutically acceptable carrier for the present compositions may include, but are not limited to, amino acids, peptides, biological polymers, non-biological polymers, simple sugars or starches, inorganic salts, and gums, which may be present singly or in combinations thereof. The peptides used in the acceptable carrier may include, e.g., gelatin and/or albumin. Cellulose or its derivatives may be used in the pharmaceutically acceptable carrier. The sugar used in the acceptable carrier may be lactose and/or glucose. Other useful sugars which may be utilized in the pharmaceutical compositions include but are not limited to, fructose, galactose, lacticol, maltitol, maltose, mannitol, melezitose, myoinositol, palatinate, raffinose, stachyose, sucrose, tehalose, xylitol, hydrates thereof, and combinations of thereof. Binders may be included in the pharmaceutically acceptable carrier. Examples of binders include, but are not limited to, starches (for example, corn starch or potato starch), gelatin; natural or synthetic gums such as acacia, sodium alginate, powdered tragacanth, guar gum, cellulose or cellulose derivatives (for example, methycellulose, ethyl cellulose, cellulose acetate); microcrystalline cellulose, polyvinyl pyrrolidone, and mixtures thereof. Inorganic salts used in the acceptable carrier may be a magnesium salt, for example, magnesium chloride or magnesium sulfate. Other inorganic salts may be used, for example, calcium salts. Examples of calcium salts include, but are not limited to, calcium chloride, calcium sulfate. Other examples of substances which may be used in the pharmaceutically acceptable carrier include, but are not limited to, vegetable oils, such as peanut oil, cottonseed oil, olive oil, corn oil; polyols such as glycerin, propylene glycol, polyethylene glycol; pyrogen-free water, isotonic saline, phosphate buffer solutions; emulsifiers, such as the Tweens®; wetting agents, lubricants, coloring agents, flavoring agents, preservatives.

The term “wetting agents” may be used interchangeably with “surfactants”, and refers to substances that lower the surface tension of a liquid, thus allowing the liquid to spread more easily. Surfactant which can be used to form pharmaceutical compositions and dosage forms of the application include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. A useful parameter that may be used to characterize the relative hydrophilicity and hydrophobicity of non-ionic amphiphilic compounds is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are generally considered to be compounds having an HLB value equal to or less than about 10. However, HLB value of a surfactant merely provides a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts, fatty acid derivatives of amino acids, glyceride derivatives of amino acids, fusidic acid salts, oligopeptides, and polypeptides, oligopeptides, and polypeptides, lecithins and hydrogenated lecithins, lysolecithins and hydrogenated lysolecithins, phospholipids and derivatives thereof, fatty acid salts, lysophospholipids and derivatives thereof, carnitine fatty acid ester salts, salts of alkylsulfates, sodium docusate, acylactylates, mono- and di-acetylated tartaric acid esters of mono- and di-glycerides, succinylated mono- and di-glycerides, citric acid esters of mono- and di-glycerides, and mixtures thereof.

Within the aforementioned group, ionic surfactants include, but are not limited to, lecithins, lysolecithin, phospholipids, lysophospholipids and derivatives thereof, carnitine fatty acid ester salts, fatty acid salts, salts of alkylsulfates, sodium docusate, acylactylates, mono- and di-acetylated tartaric acid esters of mono- and di-glycerides, succinylated mono- and di-glycerides, citric acid esters of mono- and di-glycerides, and mixtures thereof.

Ionic surfactants may be the ionized forms of lactylic esters of fatty acids, lecithin, lysolecithin, phosphatidylethanolamine, phosphatidylcholine, phosphatidylglycerol, phosphatidic acid, phosphatidylserine, lysophosphatidylcholine, lysophosphatidylserine, lysophosphatidylethanolamine, lysophosphatidylglycerol, lysophosphatidic acid, PEG-phosphatidylethanolamine, PVP-phosphatidylethanolamine, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, linoleate, linolenate, stearate, ricinoleate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides, alkylthioglucosides, alkylmaltosides, lauryl macrogolglycerides, polyoxyalkylene alkyl ethers such as polyethylene glycol alkyl ethers, polyoxyalkylene alkylphenols such as polyethylene glycol alkyl phenols, polyethylene glycol glycerol fatty acid esters, polyoxyalkylene alkyl phenol fatty acid esters such as polyethylene glycol fatty acids monoesters and polyethylene glycol fatty acids diesters, polyglycerol fatty acid esters, polyoxyethylene-polyoxypropylene block copolymers and mixtures thereof, polyoxyalkylene sorbitan fatty acid esters such as polyethylene glycol sorbitan fatty acid esters, hydrophilic transesterification products of a polyol with at least one member of the group consisting of glycerides, vegetable oils, hydrogenated vegetable oils, fatty acids, and sterols, polyoxyethylene sterols and derivatives or analogues thereof, polyoxyethylated vitamins and derivatives thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-12 oleate, PEG-15 oleate, PEG-20 oleate, PEG-20 laurate, PEG-32 dilaurate, PEG-32 laurate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-glyceryl trioleate, PEG-32 dioleate, PEG-20 glyceryl laurate, PEG-20 trioleate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-50 hydrogenated castor oil, PEG-40 castor oil, PEG-35 castor oil, PEG-60 castor oil, PEG-40 palm kernel oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phytosterol, PEG-30 soya sterol, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, but are not limited to, fatty alcohols, glycerol fatty acid esters, acetylated glycerol fatty acid esters, lower alcohol fatty acids esters, propylene glycol fatty acid esters, sorbitan fatty acid esters, polyethylene glycol sorbitan fatty acid esters, sterols and sterol derivatives, polyoxyethylated sterols and sterol derivatives, polyethylene glycol alkyl ethers, sugar ethers, sugar esters, hydrophobic transesterification products of a polyol with at least one member of the group consisting of glycerides, vegetable oils, hydrogenated vegetable oils, fatty acids and sterols, oil-soluble vitamins/vitamin derivatives, lactic acid derivatives of mono- and di-glycerides, and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

Lubricants that may be used in the pharmaceutical composition include, but are not limited to, agar, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, or mixtures thereof. Additional lubricants include, by way of example, a syloid silica gel, a coagulated aerosol of synthetic silica, or mixtures thereof. A lubricant can optionally be added, in an amount of less than about 1 weight percent of the pharmaceutical composition.

The composition may include one or more pharmaceutically acceptable additives, which may include, but are not limited to, detackifiers, anti-foaming agents, buffering agents, antioxidants, polymers, preservatives, chelating agents, odorants, opacifiers, suspending agents, fillers, plasticizers, and mixtures thereof.

In some embodiments, the pharmaceutically acceptable carrier comprises more than 90%, more than 80%, more than 70%, more than 60%, more than 50%, more than 40%, more than 30%, more than 20%, more than 10%, more than 9%, more than 8%, more than 6%, more than 5%, more than 4%, more than 3%, more than 2%, more than 1%, more than 0.5%, more than 0.4%, more than 0.3%, more than 0.2%, more than 0.1%, more than 0.09%, more than 0.08%, more than 0.07%, more than 0.06%, more than 0.05%, more than 0.04%, more than 0.03%, more than 0.02%, more than 0.01%, more than 0.009%, more than 0.008%, more than 0.007%, more than 0.006%, more than 0.005%, more than 0.004%, more than 0.003%, more than 0.002%, more than 0.001%, more than 0.0009%, more than 0.0008%, more than 0.0007%, more than 0.0006%, more than 0.0005%, more than 0.0004%, more than 0.0003%, more than 0.0002%, or more than 0.0001% of the pharmaceutical composition by w/w, w/v or v/v.

In some embodiments, the concentration of the metopimazine, or pharmaceutically acceptable salt thereof (e.g., a pharmaceutically acceptable salt of metopimazine, such as metopimazine mesylate), in the composition comprises less than 100%, less than 90%, less than 80%, less than 70%, less than 60%, less than 50%, less than 40%, less than 30%, less than 20%, less than 10%, less than 9%, less than 8%, less than 6%, less than 5%, less than 4%, less than 3%, less than 2%, less than 1%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, less than 0.01%, less than 0.009%, less than 0.008%, less than 0.007%, less than 0.006%, less than 0.005%, less than 0.004%, less than 0.003%, less than 0.002%, less than 0.001%, less than 0.0009%, less than 0.0008%, less than 0.0007%, less than 0.0006%, less than 0.0005%, less than 0.0004%, less than 0.0003%, less than 0.0002%, or less than 0.0001% of the pharmaceutical composition by w/w, w/v or v/v.

In some embodiments, the concentration of the metopimazine, or pharmaceutically acceptable salt thereof (e.g., a pharmaceutically acceptable salt of metopimazine, such as metopimazine mesylate), is in the range of about 0.0001% to about 50%, about 0.001% to about 40%, about 0.01% to about 20%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% of the pharmaceutical composition by w/w, w/v or v/v.

In some embodiments, the concentration of the metopimazine, or pharmaceutically acceptable salt thereof (e.g., a pharmaceutically acceptable salt of metopimazine, such as metopimazine mesylate), is in the range of about 0.0001% to about 5%, about 0.001% to about 4%, about 0.01% to about 2%, about 0.02% to about 1%, or about 0.05% to about 0.5% of the pharmaceutical composition by w/w, w/v or v/v.

Described below are some non-limiting examples of pharmaceutical compositions.

Pharmaceutical Compositions for Oral Administration

The pharmaceutical composition comprising an effective amount of metopimazine, or pharmaceutically acceptable salt thereof (e.g., a pharmaceutically acceptable salt of metopimazine, such as metopimazine mesylate), can be formulated for oral administration. In some embodiments, the pharmaceutical composition comprising an effective amount of metopimazine, or pharmaceutically acceptable salt thereof (e.g., a pharmaceutically acceptable salt of metopimazine, such as metopimazine mesylate), for oral administration is a solid pharmaceutical composition. In some embodiments, the solid pharmaceutical composition may be presented as discrete (e.g., unit) oral dosage forms. Non-limiting examples of discrete oral dosage forms include tablets, capsules, caplets, gelatin capsules, sustained release formulations, lozenges, thin films, lollipops, chewing gum. In some embodiments, the discrete oral dosage form is an orally disintegrating oral dosage form, such as, e.g., an orally disintegrating tablet.

Discrete oral dosage forms such as tablets may be coated by known techniques to delay or prolong absorption in the gastrointestinal tract, thus providing a sustained action of a longer period of time. In some embodiments, the metopimazine, or pharmaceutically acceptable salt thereof (e.g., a pharmaceutically acceptable salt of metopimazine, such as metopimazine mesylate), is mixed with one or more inert solid diluents, such as calcium carbonate or calcium phosphate. In some embodiments, the metopimazine, or pharmaceutically acceptable salt thereof (e.g., a pharmaceutically acceptable salt of metopimazine, such as metopimazine mesylate), is presented as soft gelatin capsules, wherein the metopimazine, or pharmaceutically acceptable salt thereof (e.g., a pharmaceutically acceptable salt of metopimazine, such as metopimazine mesylate), is mixed with water or an oil medium, such as peanut oil, or olive oil, for example.

In some embodiments, the pharmaceutical composition comprising an effective amount of metopimazine, or pharmaceutically acceptable salt thereof (e.g., a pharmaceutically acceptable salt of metopimazine, such as metopimazine mesylate), for oral administration is a liquid pharmaceutical composition. Non-limiting examples of liquid compositions for oral administration include hydrophilic suspensions, emulsions, liquids, gels, syrups, slurries, solutions, elixirs, softgels, tinctures, and hydrogels. In some embodiments, solid or liquid compositions comprising an effective amount of metopimazine, or pharmaceutically acceptable salt thereof (e.g., a pharmaceutically acceptable salt of metopimazine, such as metopimazine mesylate), for oral administration comprise various sweetening or flavoring agents, or coloring agents. Examples of coloring agents include dyes suitable for food such as those known as F.D. & C. dyes and natural coloring agents such as grape skin extract, beet red powder, beta carotene, annato, carmine, turmeric, paprika, and so forth. Derivatives, analogues, and isomers of any of the above colored compounds also may be used.

Such dosage forms may be prepared by methods well known to those skilled in the art, e.g., in a pharmacy. Such methods would comprise bringing the metopimazine, or pharmaceutically acceptable salt thereof (e.g., a pharmaceutically acceptable salt of metopimazine, such as metopimazine mesylate), into association with the pharmaceutically acceptable carrier.

This application further encompasses anhydrous pharmaceutical compositions and dosage forms comprising an effective amount of metopimazine, or pharmaceutically acceptable salt thereof (e.g., a pharmaceutically acceptable salt of metopimazine, such as metopimazine mesylate), since water may facilitate the degradation of the metopimazine, or pharmaceutically acceptable salt thereof (e.g., a pharmaceutically acceptable salt of metopimazine, such as metopimazine mesylate). In some embodiments, the anhydrous pharmaceutical compositions and dosage forms of the application are prepared using anhydrous or low moisture containing ingredients. In some embodiments, the anhydrous pharmaceutical compositions and dosage forms of the application are prepared under low humidity or low moisture conditions. The pharmaceutical compositions of the present application which contain lactose may be made anhydrous if substantial contact with moisture and/or humidity during manufacturing, packaging, and/or storage is expected. An anhydrous pharmaceutical composition comprising metopimazine, or pharmaceutically acceptable salt thereof, may be prepared and stored such that its anhydrous nature is maintained. For example, the anhydrous compositions may be packaged using materials known to prevent exposure to water such that they can be included in suitable formulary kits, examples of which include, but are not limited to, hermetically sealed foils, plastic or the like, unit dose containers, blister packs, and strip packs.

Pharmaceutical Compositions for Injection or Parenteral Administration

In some embodiments, the pharmaceutical composition is formulated for parenteral administration. “Parenteral administration” generally refers to routes of administration other than the gastro-intestinal tract. Examples of parenteral administration include, but are not limited to, intravenous injection, intra-arterial injection, intrathecal injection (into the spinal cord), intratonsillary injection, subcutaneous injection, intramuscular injection, infusion, or implantation. Infusion may be intradermal, or subcutaneous, or through a transdermal implant. Exemplary pharmaceutical compositions for parenteral administration are disclosed in the following references which are hereby incorporated by reference: U.S. Patent Application Pub. No 2006/0287221, U.S. Pat. Nos. 5,244,925, 4,309,421, 4,158,707, and 5,164,405, all of which are hereby incorporated by reference.

Compositions formulated for parenteral administration may include aqueous solutions and/or buffers commonly used for injection and/or infusion. Commonly used aqueous buffers and/or solutions may include, but are not limited to sodium chloride solutions of about 0.9%, phosphate buffers, Lactated Ringer's solution, Acetated ringer's solution, phosphate buffered saline, citrate buffers, Tris buffers, histidine buffers, HEPES buffers, glycine buffers, N-glycylglycine buffers, and the like. Other pharmaceutically acceptable carriers for parenteral administration may include ethanol, glycerol, propylene glycol, cyclodextrin and cyclodextrin derivatives, vegetable oils, and the like.

In some embodiments, pharmaceutical compositions for injection and/or infusion contain preservatives present in amounts that effectively prevent or reduce microbial contamination or degradation. Various agents, e.g., phenol, m-cresol, benzyl alcohol, parabens, chlorobutanol, methotrexate, sorbic acid, thimerosol, ethyl hydroxybenzoate, bismuth tribromophenate, methyl hydroxybenzoate, bacitracin, propyl hydroxybenzoate, erythromycin, 5-fluorouracil, doxorubicin, mitoxantrone, rifamycin, chlorocresol, benzalkonium chlorides, may be used to prevent or reduce contamination.

In some embodiments, sterile solutions are prepared by incorporating metopimazine, or pharmaceutically acceptable salt thereof (e.g., a pharmaceutically acceptable salt of metopimazine, such as metopimazine mesylate), in the required amount in the appropriate solvent with various other ingredients as described herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain methods of preparation include but are not limited to vacuum-drying and freeze-drying techniques which yield a powder of the active ingredient plus any additional desired ingredient from a previously sterile-filtered solution thereof.

In some embodiments, the pharmaceutical composition is formulated for topical and/or transdermal delivery. Compositions of the present application can be formulated into preparations in liquid, semi-solid, or solid forms suitable for local or topical administration. Examples of forms suitable for topical or local administration include but are not limited to, gels, water soluble jellies, creams, lotions, suspensions, foams, powders, slurries, ointments, oils, pastes, suppositories, solutions, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical composition may comprise suitable solid or gel phase carriers, which are compounds that allow increased penetration of, or assist in the delivery of, therapeutic molecules across the stratum corneum barrier of the skin. There are many of these penetration-enhancing molecules known to those skilled in the art of topical formulation. Examples of such carriers and excipients include, but are not limited to, alcohols (e.g., ethanol), fatty acids (e.g., oleic acid), humectants (e.g., urea), glycols (e.g., propylene glycol), surfactants (e.g., isopropyl myristate and sodium lauryl sulfate), glycerol monolaurate, sulfoxides, pyrrolidones, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present application employs transdermal delivery devices (“patches”). Such transdermal patches may be used to provide continuous or discontinuous infusion of metopimazine, or pharmaceutically acceptable salt thereof (e.g., a pharmaceutically acceptable salt of metopimazine, such as metopimazine mesylate), as described herein in controlled amounts, either with or without an additional agent. The construction and use of transdermal patches for the delivery of pharmaceutical agents is well known in the art. See, e.g., U.S. Pat. Nos. 5,023,252; 4,992,445; and 5,001,139; which are herein incorporated by reference.

In some embodiments, the application provides a pharmaceutical composition comprising an effective amount of metopimazine, or pharmaceutically acceptable salt thereof (e.g., a pharmaceutically acceptable salt of metopimazine, such as metopimazine mesylate), as described herein for transdermal delivery, and a pharmaceutical excipient suitable for delivery by inhalation. Compositions for inhalation include solutions and suspensions in pharmaceutically acceptable, aqueous or organic solvents, or mixtures thereof, and powders. The liquid or solid compositions may contain suitable pharmaceutically acceptable excipients as described herein. The compositions may be administered by the oral or nasal respiratory route for systemic effect. In some embodiments, compositions in preferably pharmaceutically acceptable solvents may be nebulized by use of inert gases. In some embodiments, nebulized solutions may be inhaled directly from the nebulizing device. In other embodiments, nebulizing device may be attached to a face mask tent or intermittent positive pressure breathing machine. Solution, suspension, or powder compositions may be administered, preferably orally or nasally, from devices that deliver the formulation in an appropriate manner.

Other Pharmaceutical Compositions

The pharmaceutical compositions and/or combinations employed in the present application may be formulated for intraocular (ophthalmic), rectal, sublingual, buccal, or intranasal (e.g., intrapulmonary) administration. Formulations suitable for intraocular administration include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w. Formulations suitable for sublingual administration, typically are formulated to dissolve rapidly upon placement in the mouth, allowing the active ingredient to be absorbed via blood vessels under the tongue. Exemplary sublingual formulations include, e.g., lozenges comprising the active ingredient in a flavored basis, usually sucrose and acacia or tragacanth; pastilles comprising the active ingredient in an inert basis such as gelatin and glycerin, or sucrose and acacia; mouthwashes comprising the active ingredient in a suitable liquid carrier; orally disintegrating tablets which may, for example, disintegrate in less than 90 seconds upon placement in the mouth; and thin films. Such disintegration can be measured by an in vitro dissolution test. Formulations for buccal administration can include, e.g., buccal tablets, bioadhesive particles, wafers, lozenges, medicated chewing gums, adhesive gels, patches, films, which may be delivered as an aqueous solution, a paste, an ointment, or aerosol, to name a few. Formulations for rectal administration may be presented as a suppository with a suitable base comprising for example cocoa butter or a salicylate. Formulations suitable for intrapulmonary or nasal administration can have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of cancerous infections as described below. A pharmacological formulation of the present application can be administered to the patient in an injectable formulation containing any compatible carrier, such as various vehicle, adjuvants, additives, and diluents; or the metopimazine mesylate utilized in the present application can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present application include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Preparations for such pharmaceutical compositions are described in, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Exemplary Modes of Administration

Administration of a pharmaceutical composition and/or combination as described herein can be performed by any method that enables delivery of the metopimazine, or pharmaceutically acceptable salt thereof (e.g., a pharmaceutically acceptable salt of metopimazine, such as metopimazine mesylate), to the site of action. The composition and/or combination may be administered orally, parenterally, enterally, intraperitoneally, topically, transdermally, ophthalmically, intranasally, locally, non-orally, via spray, subcutaneously, intravenously, intratonsillary, intramuscularly, buccally, sublingually, rectally, intra-arterially, by infusion, or intrathecally. In some embodiments, the composition and/or combination is administered orally. In some cases, the oral administration may comprise administration of any of the oral dosage forms as described herein. The effective amount of metopimazine, or pharmaceutically acceptable salt thereof (e.g., a pharmaceutically acceptable salt of metopimazine, such as metopimazine mesylate), administered will be dependent on the subject being treated, the severity of the disorder or condition, the rate of administration, the disposition of the metopimazine, or pharmaceutically acceptable salt thereof (e.g., a pharmaceutically acceptable salt of metopimazine, such as metopimazine mesylate), and the discretion of the prescribing physician.

A subject can be administered a daily dosage of metopimazine, or pharmaceutically acceptable salt thereof (e.g., a pharmaceutically acceptable salt of metopimazine, such as metopimazine mesylate), as described herein for the treatment of an enteric nervous system disorder. The daily dosage can be from about 0.01 mg/kg to about 500 mg/kg of body weight per day.

In some embodiments, administration may comprise infusion. In some cases, infusion may involve chronic, steady dosing. Devices for chronic, steady dosing, e.g., by a controlled pump, are known in the art, (examples may be described in U.S. Pat. Nos. 7,341,577, 7,351,239, 8,058,251, herein incorporated by reference).

Administration of the metopimazine, or pharmaceutically acceptable salt thereof (e.g., a pharmaceutically acceptable salt of metopimazine, such as metopimazine mesylate), may continue as long as necessary. In some embodiments, the metopimazine, or pharmaceutically acceptable salt thereof (e.g., a pharmaceutically acceptable salt of metopimazine, such as metopimazine mesylate), is administered for more than 1, 2, 3, 4, 5, 6, 7, 14, or 28 days. In particular embodiments, the metopimazine, or pharmaceutically acceptable salt thereof (e.g., a pharmaceutically acceptable salt of metopimazine, such as metopimazine mesylate), is administered for more than 5 days. In some embodiments, the metopimazine, or pharmaceutically acceptable salt thereof (e.g., a pharmaceutically acceptable salt of metopimazine, such as metopimazine mesylate), is administered for more than 12 weeks. In some embodiments, the metopimazine, or pharmaceutically acceptable salt thereof (e.g., a pharmaceutically acceptable salt of metopimazine, such as metopimazine mesylate), is administered for more than 1 month, more than 2 months, more than 4 months, more than 6 months, more than 1 year, more than 2 years, or more than 5 years. In some embodiments, the metopimazine, or pharmaceutically acceptable salt thereof (e.g., a pharmaceutically acceptable salt of metopimazine, such as metopimazine mesylate), is administered for less than five days.

Exemplary Combination Therapies

In the methods disclosed herein comprising co-administration of an additional agent, the additional agents may be: small molecules, nutraceuticals, vitamins, e.g., vitamin D, drugs, pro-drugs, biologics, peptides, peptide mimetics, antibodies, antibody fragments, cell or tissue transplants, vaccines, polynucleotides, DNA molecules, RNA molecules, (i.e.—siRNA, miRNA), antibodies conjugated to drugs, toxins, fusion proteins. Agents may be delivered by vectors, including but not limited to: plasmid vectors, viral vectors, non-viral vectors, liposomal formulations, nanoparticle formulations, toxins, therapeutic radioisotopes, etc.

In some embodiments, a method of the application comprises co-administration of a peripherally restricted dopamine decarboxylase inhibitor and a pharmaceutical composition as described herein. For example, an application method may comprise co-administration of carbidopa and a pharmaceutical composition as described herein.

The additional agent can be an agent for use in the treatment of an enteric nervous system disorder. In some embodiments, the additional agent is an additional anti-emetic agent (e.g., used for the treatment of nausea and/or vomiting). The additional anti-emetic agent can be, by way of non-limiting example only, a 5-HT3 receptor antagonist, a dopamine receptor antagonist, an NK1 receptor antagonist, an antihistamine, a cannabinoid, a benzodiazepine, an anticholinergic agent, a steroid, a phenothiazine or other anti-emetic. Exemplary 5-HT3 receptor antagonists include, but are not limited to, Odansetron, Tropisetron, Granisetron, Palonosetron, Dolasetron. Exemplary dopamine receptor antagonists include, e.g., Metoclopramide (Reglan), Domperidone (Motilium), Olanzapine (Zyprexa) Droperidol, Haloperidol, Chlorpromazine, Promethazine, Prochlorperazine, Alizapride, Prochlorperazine, Sulpiride. Exemplary NK1 receptor antagonists include, e.g., Aprepitant, Tradipitant or Casopitant. Exemplary antihistamines include, e.g., Cyclizine, Diphenhydramine (Benadryl), Dimenhydrinate (Gravol, Dramamine), Doxylamine, Meclozine (Bonine, Antivert), Promethazine (Pentazine, Phenergan, Promacot), and Hydroxyzine (Vistaril), Cimetidine, Famotidine, Lafutidine, Nizatidine, Ranitidine, Roxatidine, Tiotidine. Exemplary cannabinoids include, e.g., Cannabis, Sativex, tetrahydrocannabinol, Dronabinol, and synthetic cannabinoids such as Nabilone. Exemplary benzodiazepines include, e.g., midazolam or Lorazepam. Exemplary anticholinergic agents include, e.g., scopolamine. Other exemplary anti-emetics include, e.g., Trimethobenzamide, Ginger, Emetrol, Propofol, Peppermint, Erythromycin, Muscimol, botulinum toxin A (e.g., injected into the stomach to relax the pyloric muscle), and Ajwain. Exemplary phenothiazines include, e.g., Thiethylperazine (Torecan), Prochlorperazine (Compro, Compazine), Promethazine (Phenergan), Thiethylperazine (Torecan). Other exemplary agents are prokinetics such as Bethanechol (urecholine), Cisapride (Propulsid), Domperidone (Motilium), Erythromycin (Emycin), Metoclopramide (Reglan, Metozolv), Pyridostigmine bromide (Mestinon). The additional agent can be an agent for treatment of another disease or clinical syndrome associated with gastroparesis. Exemplary other diseases and clinical syndromes are described herein.

The additional agent can be a prokinetic agent such as a grelin agonist or a motilin agonist. Exemplary grelin agonists include e.g relamorelin and ulimorelin. Exemplary motilin agonist include, e.g. Erythromycin, Azithromycin or Clarithromycin.

The additional agent can be an agent for treatment of diabetes. Exemplary agents for the treatment of diabetes include, e.g., insulin. Other agents for the treatment of diabetes are described in, for example, U.S. Pat. Nos. 6,274,549, 8,349,818, 6,184,209, US Patent Application Publication No. US20070129307, and PCT Application Publication No. WO/2004/082667A1, all of which are hereby incorporated by reference.

The additional agent can be for treatment of upper and lower dysmotility disorders associated with Parkinson's disease. The additional agent can be for treatment of Parkinson's disease. Exemplary agents for the treatment of Parkinson's disease include, e.g., dopaminergic agents, MAO-A or B inhibitors such as, e.g., Selegiline, COMT inhibitors such as Entacapone, Amantadine, stem cell transplant, and neuroprotective agents. Exemplary dopaminergic agents include, but are not limited to levodopa, Bromocriptine, Pergolide, Pramipexole, Cabergoline, Ropinorole, Apomorphine or a combination thereof.

The additional agent can be for treatment of pain such as analgesics. Examplary analgesics include, e.g., Amitriptyline (Elavil), Gabapentin (Neurontin), Pregabalin (Lyrica), Hydromorphone (Dilaudid), Ibuprofen (Advil, Motrin), Acetaminophen (Tylanol), Ketorolac tromethamine (Toradol) Mirtazapine (Remeron), Morphine (MSCotin), Naproxen (Aleve), Nortriptyline (Pamelor), Oxycodone (Oxycotin), Oxycodone and paracetamol (Percocet), Tapentadol (Nucynta), Tramadol (Ultram, Ultracet).

The additional agent can be for treatment of hypothyroidism, hyperthyroidism, or hyperparathyroidism. Exemplary agents for the treatment of such diseases include, e.g., beta-adrenergic blockers (“beta blockers”), levothyroxine calcimimetics, estrogen, progesterone, bisphosphonates.

The additional agent can be for treatment of adrenal insufficiency. Exemplary agents for treatment of adrenal insufficiency include, e.g., corticosteroid hormones (for example, aldosterone, fludrocortisones, and cortisol).

The additional agent can be for treatment of gastroesophageal reflux. Exemplary agents for treatment of gastroesophageal reflux include, e.g., antacids such as calcium carbonate or magnesiun hydroxyde, for example, proton pump inhibitors such as Omeprazole, H2 receptor antagonists such as Ranitidine, Famotidine, Antacids, Mosapride, Sucralfate, and Baclofen, Potassium-Competitive Acid Blockers (P-CABs) such as Vonoprazan, Fexuprezan or Tegoprazan.

The additional agent can be for treatment of scleroderma. For example, the additional agent can be D-penicillamine, Colchicine, PUVA, Relaxin, Cyclosporine, and EPA (omega-3 oil derivative), immunosupressants such as, e.g., Methotrexate, Cyclophosphamide, Azathioprine, and Mycophenolate. The additional agent can be for treatment of polymyositis. For example, the additional agent can be a corticosteroid, e.g., Prednisone, or can be an immunosuppressant.

The additional agent can be for treatment of muscular dystrophy. For example, the additional agent can be, e.g., a glucocorticoid receptor antagonist. Exemplary glucocorticoid receptor antagonists include, but are not limited to, mifepristone, 11(3-(4-dimethylaminoethoxyphenyl)-17a-propynyl-17(3-hydroxy-4,9 estradien-3-one, 17(3-hydroxy-17a-19-(4-methylphenyl)androsta-4,9(11)-dien-3-one, 4a(S)-Benzyl-2(R)-prop-1-ynyl-1,2,3,4,4a,9,10,10a(R)-octahydro-phenanthrene-2,7-diol and 4a(S)-Benzyl-2(R)-chloroethynyl-1,2,3,4,4a,9,10,10a(R)-octahydro-phenanthrene-2,7-diol, and (11(3,17(3)-11-(1,3-benzodioxo-5-yl)-17-hydroxy-17-(1-propynyl)estra-4,9-dien-3-one.

The additional agent can be for treatment of amyloidosis. For example, the additional agent can be an amyloid beta sheet mimic, an antioxidant, molecular chaperone, or other agent. Exemplary agents for the treatment of amyloidosis are described in, e.g., WO/2008/141074. Exemplary molecular chaperones include, e.g., HSP60, HSP70, HSP90, HSP100, BiP, GRP94, GRP170, calnexin and calreticulin, Protein disulfide isomerase (PDI), Peptidyl prolyl cis-trans-isomerase (PPI), trimethylamine N-oxide (TMAO), betaine, glycine betaine, glycero-phosphorylcholine, carbohydrates such as, e.g., glycerol, sorbitol, arabitol, myo-inositol and trehalose, choline, 4-Phenyl butyric acid, and taurine-conjugated ursodeoxycholic acid.

The additional agent can be for treatment of chronic idiopathic pseudoobstruction. For example, the additional agent can be Prucalopride, Pyridostigmine, Metoclopramide, Cisapride, Linaclotide, Octreotide, cannabinoids, and Erythromycin.

The additional agent can be for treatment of dermatomyositis. For example, the additional agent can be Prednisolone, Methotrexate, Mycophenolate (CellCept/Myfortic), intravenous immunoglobulins, Azathioprine (Imuran), Cyclophosphamide, Rituximab, and Acthar Gel.

The additional agent can be for treatment of systemic lupus erytematosus. For example, the additional agent can be renal transplant, corticosteroids, immunosupressants, Hydroxychloroquine, Cyclophosphamide, Mycophenolic acid, immunosupressants, analgesics, intravenous immunoglobins, and the like.

The additional agent can be for treatment of anorexia and/or bulimia. For example, the additional agent can be olanzapine, a tricyclic antidepressant, an MAO inhibitor, Mianserin, a selective serotonin reuptake inhibitor, e.g., Fluoxetine, ithium carbonate, Trazodone, and Bupropion, Phenytoin, Carbamazepine, and valproic acid, opiate antagonists such as, e.g., Naloxone and Naltrexone, and Topiramate.

The additional agent can be for treatment of depression. For example, the additional agent can be a selective serotonin reuptake inhibitor, a serotonin and norepinephrine reuptake inhibitor, Bupropion, a tricyclic antidepressant, a monoamine oxidase inhibitor, and the like. The additional agent can be for treatment of paraneoplastic syndrome. The additional agent can be for treatment of a high cervical cord lesion. For example, the additional agent can be a corticosteroid or other anti-inflammatory medication. The additional agent can be for treatment of multiple sclerosis. For example, the additional agent can be interferon beta-lb, interferon beta-la, Glatiramer acetate, Mitoxantrone, natalizumab, Fingolimod, Teriflunomide, or Cladribine.

The additional therapeutic agent can be selected from the group consisting of serotonin agonists, serotonin antagonists, selective serotonin reuptake inhibitors, anticonvulsants, opioid receptor agonists, bradykinin receptor antagonists, NK receptor antagonists, adrenergic receptor agonists, benzodiazepines, gonadotropin-releasing hormone analogues, calcium channel blockers, and somatostatin analogs.

Dosages of the additional agent and of the metopimazine, or pharmaceutically acceptable salt thereof (e.g., a pharmaceutically acceptable salt of metopimazine, such as metopimazine mesylate), for use in the treatment of an enteric nervous system disorder can vary depending on the type of additional therapeutic agent employed, on the disease or condition being treated and so forth. Sub-therapeutic amounts of one or both of the additional agent and the metopimazine, or pharmaceutically acceptable salt thereof (e.g., a pharmaceutically acceptable salt of metopimazine, such as metopimazine mesylate), can be used. The sub-therapeutic amount of one or both of the additional agent and the metopimazine, or pharmaceutically acceptable salt thereof, can be a synergistically effective amount. Therapeutically effective amounts of one or both of the additional agent and the metopimazine, or pharmaceutically acceptable salt thereof (e.g., a pharmaceutically acceptable salt of metopimazine, such as metopimazine mesylate), can be used. The pharmaceutical composition as described herein and the additional agent may be administered either simultaneously or sequentially. If administered sequentially, the attending physician or caretaker can decide on the appropriate sequence of administering the metopimazine, or pharmaceutically acceptable salt thereof (e.g., a pharmaceutically acceptable salt of metopimazine, such as metopimazine mesylate), and the additional therapeutic agent.

In some embodiments, a method comprising administering any of the pharmaceutical compositions and/or combinations described herein further comprises combination therapy with an additional therapeutic regimen. The additional therapeutic regimen can comprise implantation of a medical device. The medical device can be implanted in the stomach and/or abdomen, e.g., in the duodenum. The medical device can be an electrical device. The medical device can be a pacemaker. Such a pacemaker can utilize electrical current to induce stomach and/or duodenal contractions, thereby promoting gastrointestinal motility. Such medical devices, and methods of using them, are disclosed in U.S. Pat. No. 8,095,218, hereby incorporated by reference.

Embodiments of the application are further described in detail by reference to the following examples. These examples are provided for the purpose of illustration only, and are not intended to be limiting unless otherwise specified. Thus, the application should in no way be construed as being limited to the following examples, but rather, should be construed to encompass any and all variations which become evident as a result of the teaching provided herein.

### EXAMPLES

The following examples are offered to illustrate but not to limit the application.

### Example 1: In Vitro Studies

Test Article, Metabolites and Reagents

Metopimazine (MPZ), metopimazine acid (MPZA), and metopimazine sulfate (MPZS) were supplied by Pacific Pharmaceutical Services (Reno, NV). Bupropion, dextromethorphan, diclofenac, midazolam, paclitaxel, phenacetin, testosterone, itraconazole, montelukast, quinidine, sulfaphenazole, benzydamine, bis(4-nitrophenyl) phosphate (BNPP), 7-ethoxycoumarin (7-EC), menadione, methimazole, phthalazine, and β nicotinamide adenine dinucleotide phosphate (NADPH, tetra sodium salt) were purchased from Sigma-Aldrich (St. Louis, MO). Chlorzoxazone and 1 aminobenzotriazole (ABT) were purchased from Tokyo Chemical Industry (Portland, OR). Coumarin was purchased from Acros Organics (Waltham, MA). (S) Mephenytoin, (+)-benzylnirvanol, and tranylcypromine were purchased from Toronto Research Chemicals (North York, Ontario, Canada). Furafylline was purchased from Cayman Chemical (Ann Arbor, MI). Pooled human liver microsomes (HLM) and human kidney microsomes (HKM) were purchased from BioIVT (Westbury, NY). Pooled human liver S9 and human intestine S9 fractions and pooled human liver cytosol (HLC) were purchased from Xenotech (Lenexa, KS). Supersomes™ CYP1A2, CYP2A6, CYP2B6, CYP2C8, CYP2C9, CYP2C19, CYP2D6, CYP2E1, CYP3A4, CYP3A5, FMO1, FMO3, and FM05, were purchased from Corning Inc. (Corning, NY). CTL P450 (Supersomes™ containing only P450 reductase and cytochrome b5) was also purchased from Corning, Inc., and used to normalize the protein content in the CYP reactions. Upon receipt, all test systems were stored at <70° C. until use.

Preparation of Reagents

Monobasic potassium phosphate (1 M) and dibasic potassium phosphate (1 M) stock solutions were diluted with water (Milli-QC), Millipore) to 100 mM. The 100 mM potassium phosphate buffer was prepared by adding the monobasic potassium phosphate solution (100 mM) to dibasic potassium phosphate solution (100 mM) until the pH value adjusted to 7.4, 8.4 or 9.4. The resulting 100 mM potassium phosphate buffer (KPi) was stored refrigerated (4° C.). The NADPH solution (tetra sodium salt) was prepared fresh in KPi buffer prior to use and used in the reactions at a final concentration of 1 mM.

Preparation of Stock Solutions

Stock solutions of MPZ at 20 mM (Lot No. AP237759063 076-01) were prepared in DMSO and stored at −20° C. until use. Prior to incubations, 1 or 10 μM of MPZ (final concentration in the incubation mixture) was prepared. Primary stock solutions (Stock A) of positive control probe (7-EC) were prepared in ethanol and stored at −20° C. until use. The concentration of Stock A was 100 mM. On the day of the experiment, subsequent working solution of the positive control was prepared (from Stock A) at 100x the incubation concentration using appropriate buffer (Stock B).

Primary stock solutions (Stock A) of positive control probe (benzydamine and phthalazine) were prepared in DMSO and stored at −20° C. until use. The concentration of Stock A was 100 mM (for benzydamine) and 1 mM (for phthalazine), respectively. On the day of the experiment, subsequent working solution of the positive control was prepared (from Stock A) in KPi buffer (Stock B).

Incubation of MPZ in Human liver S9

Initial experiment was conducted with S9 fractions from liver and intestine to assess the role of liver and intestinal enzymes in the metabolism of MPZ. The reaction mixture contained 2.5 μM MPZ, pooled human liver or intestine S9 fraction (1 mg/mL S9 protein), 100 mM KPi buffer (pH 7.4). The reaction mixture was pre-incubated for 3-5 min at 37° C. in a humidified incubator filled with 5% CO₂ followed by the addition of NADPH (1 mM) to initiate the reaction. The reaction was incubated for 0 and 60 min. The total percentage of organic solvent in incubation mixture was <0.1%. The reactions were terminated by the addition of sample mixture of acetonitrile and ethanol (90:10, v/v). The positive control substrate, 7-EC, was incubated in triplicate in an identical manner as MPZ for S9 fractions.

Incubation with HLM

For protein concentration optimization, MPZ at 1 μM was incubated with varying concentration of HLM proteins (0.1, 0.25, 0.5, and 1.0 mg/mL microsomal protein), 100 mM KPi buffer (pH 7.4). After pre-incubation for 3-5 minutes at 37° C., the reaction was initiated by the addition of NADPH (1 mM) for 60 minutes in triplicates.

For incubation time optimization, MPZ at 1 μM was incubated with pooled HLM (0.5 mg/mL microsomal protein) and 100 mM KPi buffer (pH 7.4). After pre-incubation for) for 3-5 minutes at 37° C., the reaction was initiated by the addition NADPH (1 mM) incubated for 0, 10, 20, 40, 60, and 90 minutes to determine the optimal incubation time (linear range). All metabolic reactions in this study were terminated by the addition of chilled organic solvent (acetonitrile:ethanol 90:10, v/v) containing internal standards, unless otherwise indicated.

HLM Incubation with CYP Inhibitors

To determine the role of microsomal CYP enzymes in the metabolism of MPZ, MPZ was incubated with pooled HLM at 1 and 10 μM in triplicate and the disappearance of MPZ and the formation of known metabolites (MPZA, MPZS, and MPZH) was assessed. Initially, two sets of incubations were conducted with HLM (0.5 mg/mL microsomal protein), 1 and 1011M MPZ, 100 mM KPi buffer (pH 7.4) where one set was in the presence and the other set was in the absence of 1 mM ABT, a non-specific CYP inhibitor for 60 minutes. This allowed the assessment of total CYP contribution for MPZ metabolism. Then, CYP-specific chemical inhibitors were used to assess the contribution of individual major CYP-isoforms where HLM were incubated with 1 or 1011M MPZ in the presence and in the absence of each CYP specific inhibitor at 37° C. for 60 minutes. The following CYP-isoform specific chemical inhibitors were used; furafylline (10 μM), tranylcypromine (10 μM), montelucast (0.5 μM), sulfaphenazole (5 μM), (+) benzylnirvinol (5 μM), quinidine (1 μM) and itraconazole (2.5 μM), for CYP1A2, CYP2B6, CYP2C8, CYP2C9, CYP2C19, CYP2D6 and CYP3A4/5. Parallel incubation with seven CYP specific probe substrates were included in as positive controls. For negative control reaction, MPZ was incubated at 37° C. in duplicate in reaction mixture devoid of NADPH. All other procedures were the same as previously described.

Incubation with Recombinant CYP-isoforms

Recombinant CYP isoforms (Supersomes™) were used to determine the contribution of CYP isoforms in the metabolism of MPZ. MPZ at 1.0 μM was incubated in triplicate with individual CYP Supersomes™ (CYP 1A2, CYP2A6, CYP2B6, CYP2C8, CYP2C9, CYP2C19, CYP2D6, CYP2E1, CYP3A4, and CYP3A5). The final P450 content in the incubation mixtures was 50 pmol/mL. Protein concentrations for all CYP isoforms were normalized to the recombinant CYP containing the highest protein concentration. The reaction mixture containing MPZ or CYP substrates, KPi buffer (pH 7.4), and CYP-enzyme was pre-incubated at 37° C. for 3-5 minutes. All other procedures were the same as described previously. The marker substrates (coumarin for CYP2A6, chlorzoxazone for CYP2E1, midazolam for CYP3A4 and CYP3A5 and probe substrates for other CYP isoforms same as indicated in section 7.2.8) were incubated at 1.0 μM in parallel. The negative control for incubation reactions incubated in the absence of NADPH under identical conditions.

Incubation with HLM and HKM for FMO

MPZ, at 1.0 and 10.0 μM, was incubated with HLM or HKM to determine the contribution of microsomal FMOs on the metabolism of MPZ.

A reaction mixture containing KPi buffer (pH 8.4), HLM or HKM (0.5 mg/mL protein) and MPZ (1.0 and 10.0 μM) was pre-incubated for 3-5 minutes at 37° C. The reaction was initiated by the addition of NADPH into the reaction mixture. Reaction was conducted for 0 and 60 min. A positive control (benzydamine, 100 μM) was incubated in duplicate for 0 and 20 minutes. Parallel incubation was conducted by adding 1 mM methimazole, a non-specific FMO inhibitor, under identical conditions. Heat inactivated microsomes (50° C. for at least 5 minutes) was incubated under identical conditions negative control. All other procedures were the same as described previously.

Incubation with Recombinant FMO

MPZ at 1.0 μM was incubated in triplicate with individual recombinant FMO (Supersomes™ for FMO 1, FMO3, and FMO5). Protein concentrations for all rFMO isoforms were 0.05 mg/mL. A positive control (benzydamine, 100 μM) was incubated for 20 minutes. The reaction mixture containing MPZ or FMO substrate, KPi buffer (pH 8.4), and FMO Supersomes™ was pre-incubated at 37° C. for 3-5 minutes. All other procedures were the same as described previously. The negative control reactions were incubated absence of NADPH.

Incubations with HLM for Microsomal Hydrolase/Amidase Activity Prior to the reaction for amidase activity, the reaction mixture containing KPi buffer (pH 7.4, 8.4, or 9.4) and HLM was pre-incubated at 50° C. for 5 minutes to inactivate CYP and FMO activities in HLM. The mixture was placed on ice to cool down. An aliquot was taken out which was considered as t=0 min incubation. Then 10 μM MPZ was added into the remaining mixture and incubated at 37° C. on an orbital shaker for 60 minutes. A parallel set of incubations was conducted with 10 μM MPZ in the presence at 2 and 16 μM, of BNPP (a known inhibitor of amidase). All reactions were in triplicate. The total percentage of organic solvent in incubation mixture was ≤0.1%. The reactions were terminated by the addition of chilled acetonitrile: methanol, 90:10, (v/v). The samples were vortex-mixed and stored at ≤−70° C. and were subjected analyzed by LC MS/MS.

Incubations with HLC Fractions for AO Activity

The metabolism of MPZ was evaluated in the human liver cytosolic incubation. A metabolic incubation was performed with HLC (1 mg/mL protein) and 10 μM MPZ at 37° C. for on an orbital shaker for 60 min. A parallel set of incubations were also conducted 10 μM MPZ in the presence of 100 μM menadione, a known inhibitor of human aldehyde oxidase. Another set of incubation was also conducted 1 μM phthalazine, a known substrate for aldehyde oxidase as positive control, under identical conditions.

LC/MS/MS Method

LC separation conditions were developed for MPZ and its metabolites (MPZA and MPZS), 7 hydroxycoumarin, each metabolite of CYP probe substrate, benzydamine N oxide, and phthalazine. The LC MS/MS system consisted of a Shimadzu Nexera X2 LC-30 UPLC system (Shimadzu Corporation, Kyoto, Japan), and an API 4000 triple quadrupole mass spectrometer (SCIEX, Framingham, MA) was used for analysis. All analytes were analyzed using electrospray ionization (ESI) in positive ion mode except for 7-EC, which used negative ion mode. The LC-MS/MS system was controlled by Analyst software (version 1.6.1, SCIEX, Framingham, MA).

For each analytical method, a calibration curve was generated for the quantification of MPZ and its two metabolites (MPZA and MPZS). Due to lack of the reference standard, the hydroxylated metopimazine (MPZH) was determined qualitatively based on peak area ratio (PAR) of presumed hydroxylated analyte to the IS. PAR was also used for all positive control compounds.

Data Analysis

The degree of disappearance of each analyte was evaluated by comparing the PAR of analyte to IS or concentration in different sampling time points to the PAR of time zero, (t=0, 100%). The percent of parent remaining values were calculated according the following equation:

% parent remaining=(mean concentration or PAR at each sampling time point/mean concentration or PAR of time zero)×100

All statistics (e.g., mean, SD) presented in the data tables are based on “precision as displayed” numbers (MS Excel, version 2010). GraphPad Prism (version 8, San Diego, CA) was used for figure generation.

Results

Metabolism in Liver and Intestinal S9 Fraction, and Microsomes

Upon incubation of MPZ with HLM, three primary metabolites were found on full scan LC-MS analysis. Based on peak area, the largest signal was found at m/z 447.14, which corresponded to MPZA, the major in vivo metabolite in humans. Two other isobaric peaks were found at m/z 462.15, consistent with MPZH and MPZS metabolites (Table 1).

TABLE 1
In vitro metabolites found following metopimazine
incubations in human liver microsomes.

| | | Observed | Observed | Peak | Peak Area |
|---|---|---|---|---|---|
| | Likely | Precursor | Retention | Area at | at Time 90 |
| Compound | Metabolite | Ion (m/z) | Time (min) | Time 0 | min |
| MPZ - | — | 446.15 | 4.01 | 2373 | 715 |
| parent | | | | | |
| | MPZH | 462.15 | 2.80 | 0 | 100 |
| | MPZS | 462.15 | 3.15 | 0 | 59 |
| | MPZA | 447.14 | 4.26 | 0 | 208 |

In human liver S9 fractions, approximately 30% of MPZ disappeared and all three metabolites (MPZA, MPZH and MPZS) were formed after a 60-minute incubation. In contrast, human intestine S9 fractions showed virtually no metabolism of MPZ, indicating intestinal metabolism is unlikely. HLM incubations were optimized for protein concentration and time based on the disappearance of MPZ and determined based on PAR. MPZ (111M) was incubated with indicated HLM for 60 min. An HLM protein concentration of 0.5 mg/mL were chosen for time optimization incubation. MPZ was incubated in HLM at 0.5 mg/mL protein concentration over 90 min. MPZ disappearance was essentially linear over the incubation period and there were 37.2% remaining at 60 min incubation time, which was considered optimal for future incubations. Authentic standards of MPZ, MPZA and MPZS were available for quantification, while MPZH could only be semi-quantified using peak area ratio due to lack of a synthetic standard.

Cytochrome P450 Involvement

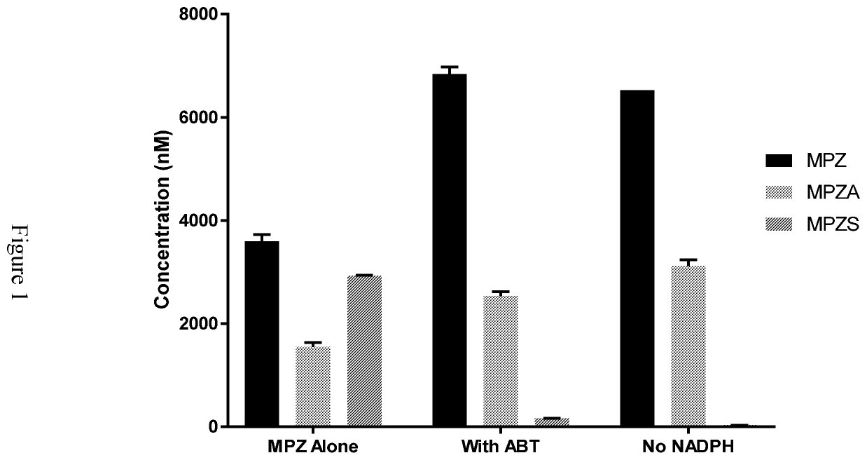

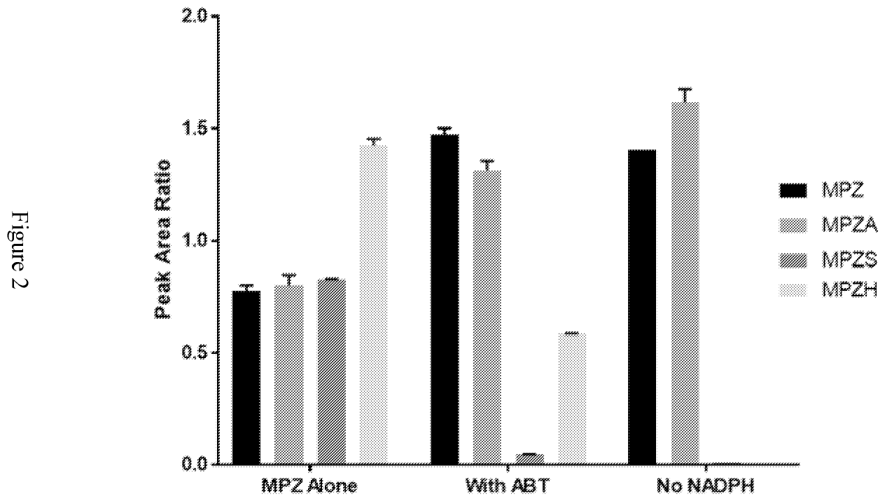

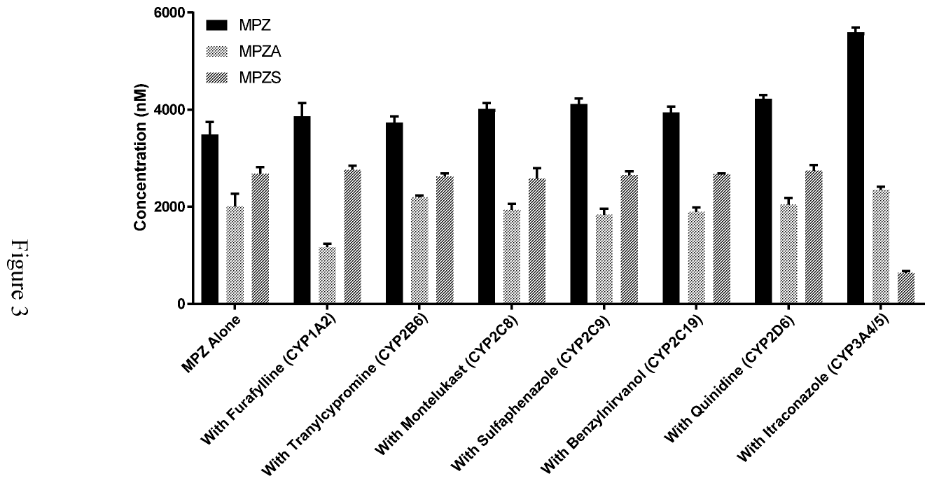

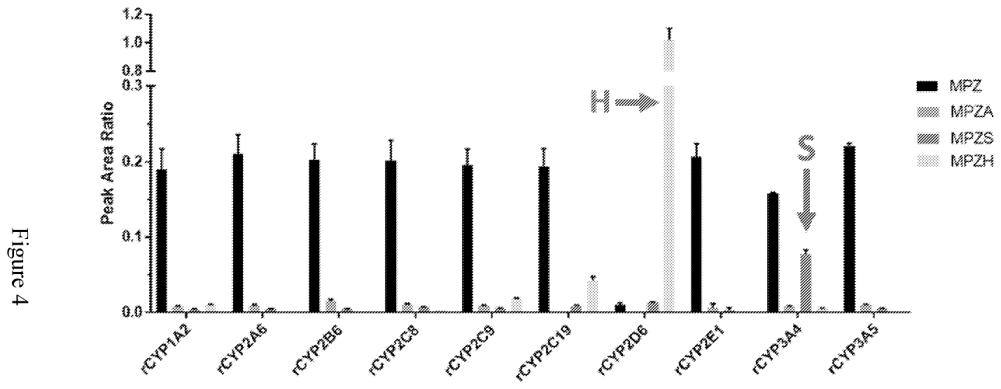

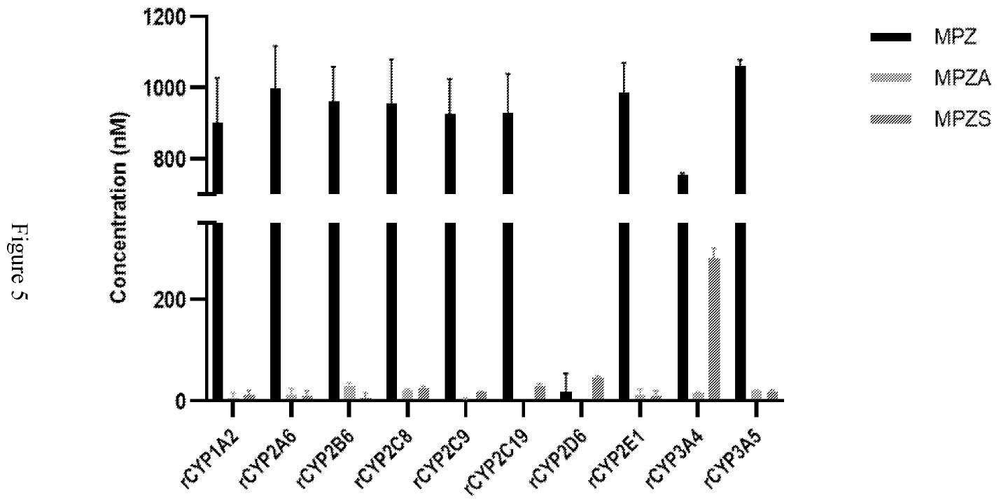

To determine the involvement of cytochrome P450 enzymes, MPZ was incubated in HLM at 1 and 1011M in the presence and absence of 1-ABT, a known non-specific CYP inhibitor, with and without NADPH cofactor addition to the incubation mixture. As shown FIG. 1, the presence of 1-ABT in the incubation inhibited the formation of MPZS but had no effect on MPZA. In addition, the absence of NADPH also did not reduce the formation of MPZA, but completely shut down the formation of MPZS. Looking qualitatively at peak area ration (PAR) in FIG. 2, MPZH formation, like MPZS, was also inhibited by the pan-CYP inhibitor 1-ABT and formation of MPZH was also completely shut down in the absence of NADPH. This indicates that NADPH-dependent cytochrome P450 enzymes are involved in the production of both the MPZS and MPZH metabolites; however, MPZA is not formed by cytochrome P450 enzymes, or any NADPH-dependent metabolic processes. To determine which CYP isoforms were involved in MPZ metabolism, separate HLM incubations were performed with specific inhibitors for each of the seven cytochrome P450 enzymes and the amount of MPZ, MPZA and MPZS quantified by mass spectrometry analysis. MPZS formation was clearly inhibited by the presence of the CYP3A4-specific inhibitor itraconazole (FIG. 3). MPZS formation was clearly inhibited by the presence of the CYP3A4-specific inhibitor itraconazole, while the CYP2D6-specific inhibitor quinidine inhibited formation of MPZH. None of these inhibitors affected MPZA formation. In addition, recombinant CYPs were incubated with MPZ for 60 min to confirm production of any metabolites as shown in FIGS. 4 and 5. In the recombinant CYP experiments, MPZS is mainly produced by CYP3A4 but also CYP2D6 to a lesser extent. Consistent with the inhibition experiments, MPZH was only produced with recombinant CYP2D6. None of the other CYP enzymes appeared to have significant contribution to MPZ metabolism.

Enzymes Involved in Formation of MPZA

Since MPZA formation was not mediated by the Cytochrome P450, other enzymes, FMO and hydroxylase, were investigated. To determine the contribution of microsomal FMOs on the metabolism of MPZ, incubations of MPZ were done in mixed, pooled HLM or HKM at 1 and 10 μM in triplicate in the presence of NADPH at pH 8.4 (a pH that should minimize CYP involvement) with and without an FMO inhibitor (methimazole). An HLM control was included in which HLM was pre-incubated at 50° C. for 5 minutes to inactivate CYP and FMO activities. Considerable formation of MPZA was observed with all conditions in HLM at pH 8.4 and was increased with increasing concentration of MPZ.

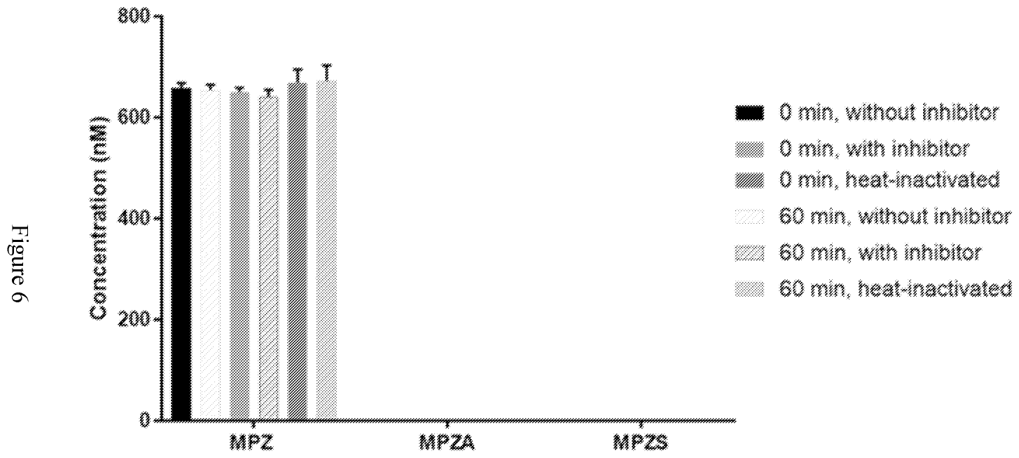

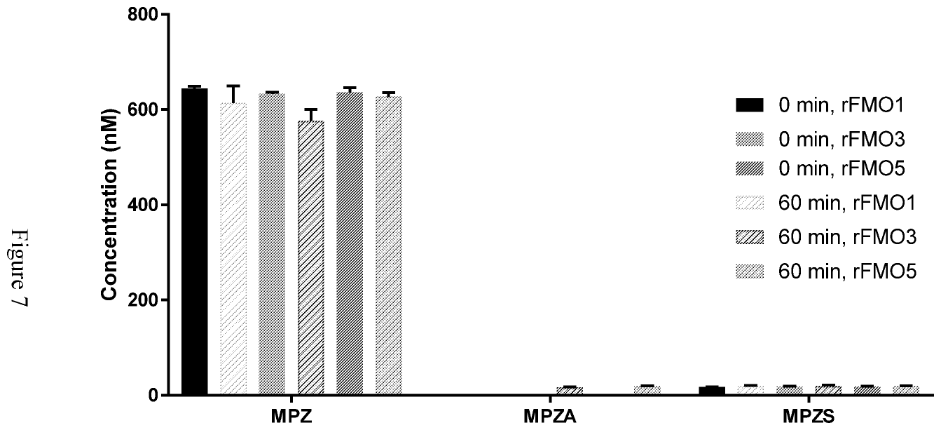

Incubation of MPZ with HKM at pH 8.4 did not produce any metabolites (FIG. 6). Together, these results indicate that liver metabolism is unaffected by pH, FMO-inhibition and even high temperature incubation, while there is no evidence of metabolism by HKM. The lack of formation of any metabolites at pH 8.4 in the HKM incubations demonstrates that the formation of MPZA is not catalyzed by pH alone. Furthermore, MPZA formation was not inhibited by a known FMO inhibitor, methimazole or heat (FMO inactivated), ruling out the involvement of FMO enzymes in the MPZA metabolite formation. Consistent with this, recombinant flavin monooxygenases enzymes (rhFMO1, rhFMO3, and rhFMO5) did not metabolize MPZ upon incubation (FIG. 7).

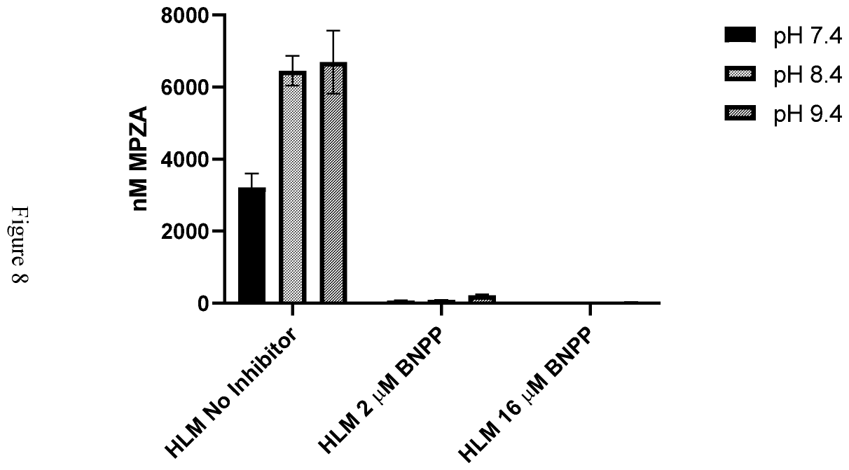

In order to track down what hydrolase activity was responsible for the metabolism of MPZ to MPZA, an HLM incubation was performed under previous conditions except at increasing pH values 7.4, 8.4 and 9.4. MPZA formation was actually faster the higher the pH, including very strong formation at pH 9.4, consistent with amidase activity (FIG. 8). In addition, bis-p-nitrophenyl phosphate (BNPP), an amidase inhibitor, was also added to some of the incubations. When this amidase inhibitor was present, at least 97% of all activity was inhibited at 2 μM, even at the highest and most active pH conditions. When this inhibitor concentration was increased to 16 μM, >99% of MPZA formation was inhibited at all three pH values. Taken together, these results are consistent with MPZA being formed by a human microsomal liver amidase.

The formation of MPZA was also observed in the incubation of 10 μM MPZ in human liver cytosol (HLC). HLC catalyzed the formation of some MPZA over 60 min of incubation, which was inhibited (48%) when 10011M menadione, a known inhibitor of human AO, was included in the incubation (Table 2). This finding suggests that human liver cytosolic aldehyde oxidase is a potential contributor to the MPZA pathway.

TABLE 2
Concentrations of Metopimazine Acid in Pooled Human Cytosolic
Incubations Without and With Menadione (AO Inhibitor)

| | Metopimazine Acid Formation | | |
|---|---|---|---|
| Incubation | (nM) | | |
| time (min) | No Menadione | 100 μM Menadione | % Inhibited |
| 0 | 0.00 | 0.00 | 47.59% |
| 60 | 1371.66 ± 70.93 | 718.90 ± 21.40 | |

BQL: Below quantifiable limit

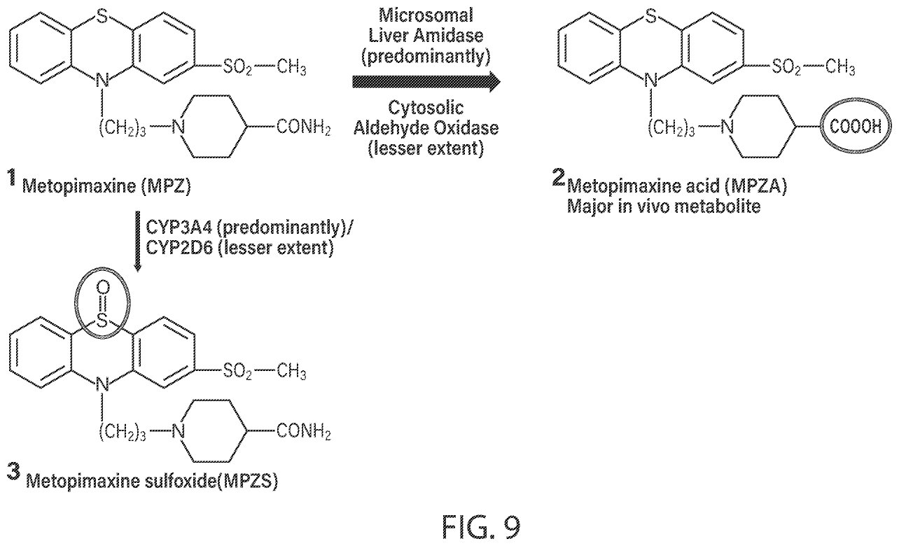

These results clearly demonstrate a plausible MPZ metabolic scheme in which the major in vivo metabolite, MPZA, is catalyzed by a liver amidase with some potential contribution from aldehyde oxidase. The minor metabolites are catalyzed by CYP3A4 and CYP2D6; however, CYP enzyme involvement was much less than microsomal hydrolase/amidase in MPZ metabolism in vitro (FIG. 9).

MPZ was shown to undergo hydrolysis in human liver microsomes to form MPZA. This conversion of MPZ to MPZA is not inhibited by any individual CYP inhibitor, including ABT, a non-specific inhibitor of CYPs. This activity is not dependent on NADPH and increases under conditions above physiologic pH. Amidase/hydrolases, such as fatty acid amide hydrolase (FAAH) are known to have optimal activity in the pH range of 8.5-10 (Ueda, N, Yamanaka, K, and Yamamoto, S (2001) Purification and Characterization of an Acid Amidase Selective for N-Palmitoylethanolamine, a Putative Endogenous Anti-inflammatory Substance J. Biol. Chem. 276: 35552-35557; Bisogno, T, Maurelli, S, Melck, D, De Petrocellis, L, and Di Marzo, V (1997) Biosynthesis, Uptake, and Degradation of Anandamide and Palmitoyl ethanolamide in Leukocytes J. Biol. Chem. 272: 3315-3323; Asano, Y, Achibana, M T, and Ani, YT, Yamada, H (1982) Purification and Characterization of Amidase which Participates in Nitrile Degradation. Agric. Biol. Chem., 46 (5): 1175-1181). In HLM, the microsomal conversion of MPZ to MPZA was maximal at the highest pH tested, pH 9.4. These characteristics clearly point to a liver amidase as the enzyme responsible for MPZA formation. Consistent with these findings, the addition of BNPP, a known amidase inhibitor (Shih, T, Pai, C, Yang, P, Chang, W, Wang, N, and Hu, O Y (2013) A Novel Mechanism Underlies the Hepatotoxicity of Pyrazinamide. Antimicrob. Agents Chemother. 57: 1685-1690; Sarich T. C., Adams, S. P., Petricca, G. and Wright, J. M. (1999) Inhibition of Isoniazid-Induced Hepatotoxicity in Rabbits by Pretreatment with an Amidase Inhibitor. J. Pharmacol. Exp. Ther. 289: 695-702), dramatically inhibited the formation of MPZA in HLM.

While liver amidase conversion appears to be the primary biotransformation enzyme involved in MPZ metabolism, another enzyme that may contribute to the formation of MPZA is the cytosolic enzyme AO. We observed the conversion MPZ to MPZA in HLC, though to a much lesser extent than HLM. This activity was moderately inhibited by menadione, a known inhibitor of AO, suggesting the possible contribution of AO to MPZ metabolism. AO is a metalloenzyme that contains molybdenum cofactor and typically shows hydroxylase activity (Mendel R R (2009) Cell biology of molybdenum. Biofactors 35: 429-434). It is most extensively expressed in the liver but also in the GI tract as well as the kidney, lungs, and skin (Abbasi A, Paragas E M, Joswig-Jones C A, Rodgers J T, Jones J P (2019) Time Course of Aldehyde Oxidase and Why It Is Nonlinear. Drug Metab Dispos 47: 473-483). AO catalyzes the oxidation of a wide range of aldehydes to their corresponding carboxylic acids. AO is involved in the metabolism of several clinically significant drugs such as famciclovir, zaleplon, zonisamide, and ziprasidone. Association of AO with pathophysiology of a number of clinical disorders such as amyotrophic lateral sclerosis and alcohol induced liver injury, has been suggested (Kitamura, S, Sugihara, K and Ohta, S (2006) Drug Metabolizing Ability of Molybdenum Hydrolases. Drug Metab. Pharmacokinet. 21 (2): 83-98).

### Example 2: In Vivo Studies

Test Article, Metabolites and Reagents; Preparation of Reagents and Stock Solutions

Supply and preparation of test articles, reagents, and solutions were as described above in Example 1.

Clinical Study

The clinical study was conducted at Biotrial Rennes (Rennes, France), in accordance with the Declaration of Helsinki. The study (NG100-101) was a double-blind, placebo-controlled, ascending single- and multiple-dose study of the safety, tolerability, and pharmacokinetics of metopimazine administered orally to healthy adult subjects. The study protocol, informed consent form, and appropriate related documents were approved by the institutional review board, and all subjects provided written informed consent prior to participation in the study.

In this study, sequential ascending single doses were administered to 4 cohorts of 8 participants (6 actives and 2 placebos per cohort, in cohorts 1, 2 and 3 following a randomization 6:2 and in cohort 4 with a 1:1 randomization for the two sentinel participants. In the morning, after at least 10 hours of fasting overnight, eight healthy adult subjects (male or female aged 18 to 45), were orally administered metopimazine (or a matching placebo). Cohort 1-4 received a matching placebo or ascending doses of Vogalene (metopimazine) capsules at the doses of 15 mg, 30 mg, 45 mg, or 60 mg, respectively. Blood samples were collected at the following time points: predose and 0.25, 0.5, 0.75, 1, 1.25, 1.5, 2, 3, 4, 6, 8, 12, 18, and 24 hours postdose; plasma samples were obtained by centrifugation of the blood samples. A comprehensive safety and pharmacokinetic analysis was performed for each cohort for the SAD and MAD parts of the study and will be presented elsewhere. A fit-for-purpose discovery method was used to analyze 4 subjects from the highest single dosed cohort for the presence of metabolites. Plasma concentrations of metopimazine, metopimazine acid and metopimazine sulfoxide were quantified using LC-MS/MS methods.

Rat PK Study

The pharmacokinetics of metopimazine (parent) and metopimazine acid (metabolite) were evaluated in male and female Sprague Dawley rats following a single oral dose of 45 mg/kg metopimazine (free base). Three animals per sex in each dose group were sampled at each timepoint after administration (0.25, 0.5, 1, 2, 4, 8, 12, and 24 hours). The rats (Rattus norvegicus) were obtained from Envigo RMS, Inc. (Indianapolis, IN) and were 2 months old. Animals were weighed prior to each dose administration for the purpose of dose calculation. Whole venous blood samples of approximately 0.5 mL were collected from a peripheral vein of animals for determination of exposure to metopimazine and metopimazine acid. Three animals per sex were sampled at each timepoint. Samples were collected in tubes containing K2EDTA with protease inhibitor cocktail (Sigma, #P8340, at ratio 1:100) and placed on ice until processed for plasma by centrifugation under refrigeration for at least ten minutes at 3000 rpm. After centrifugation, supernatant was removed and stored frozen in a −70° C. freezer until analyzed by LC-MS/MS.

Dog PK Study

The pharmacokinetics of metopimazine (parent) and metopimazine acid (metabolite) were evaluated in three male, beagle dogs following a single oral dose of 10 mg/kg metopimazine (free base). The dogs were obtained from Marshall BioResources (North Rose, NY). Animals were weighed prior to each dose administration for the purpose of dose calculation. Metopimazine was administered in size 13 gelatin capsules. Whole venous blood samples of approximately 1.0 mL were collected from a peripheral vein of all animals for determination of metopimazine and metopimazine acid. Samples were collected at the following timepoints: 0.25, 0.5, 1, 2, 4, 8, 12, and 24 hours after test article administration. All animals were sampled at each timepoint. Samples were collected in tubes containing K2EDTA and placed on ice until processed for plasma by centrifugation under refrigeration for at least ten minutes at 3000 rpm. After centrifugation, supernatant was removed and stored frozen in a −70° C. freezer until analyzed by LC-MS/MS.

Preparation of Human Plasma Samples

Venous blood samples (6 mL) for the determination of plasma concentrations of MPZ and metabolites (MPZA and MPZS) were drawn by direct venipuncture or via an intravenous catheter into K2EDTA Vacutainer® tubes. It was important to avoid hemolysis during blood collection. After collection, the blood samples were centrifuged within 60 minutes, at 3000 rotations per minute for 10 minutes at 5° C. The resulting plasma was separated into 2 equal aliquots of 1000 μL (aliquots A & B) and transferred to polypropylene tubes, which were labelled, frozen within 120 minutes of blood collection and stored at −70° C. until required for bioanalysis. Immediately prior to bioanalysis, samples were thawed and a 200 μL aliquots were removed and analytes extracted using acetonitrile protein precipitation. The extracted samples were analyzed twice, once for quantification of MPZ and MPZA and again for MPZS quantification as described below.

LC/MS/MS Method for Metopimazine and Metopimazine Acid in Rat, Dog, and Human Plasma Samples

An MRM LC-MS/MS method was developed for MPZ and metopimazine acid (MPZA) in rat, dog, and human plasma. The method was run in reverse phase in positive ion mode. Using an LC-MS/MS system that consisted of a Shimadzu Nexera X2 LC-30 UPLC system (Shimadzu Corporation, Kyoto, Japan), and an API 4000 triple quadrupole mass spectrometer (SCIEX, Framingham, MA) running Analyst software 1.4. LC separation conditions were achieved for MPZ and MPZA using a Supelco Discovery HS F5 50×4.0 mm 3 μm column (Sigma-Aldrich, St. Louis, MO). D6-isotopically labeled internal standards were used for each analyte. The following m/z transitions were monitored: metopimazine 446.3→141.1 and metopimazine acid 447.2→142.1. A calibration curve was generated for the quantification of MPZ and MPZA.

LC/MS/MS Method for Metopimazine Sulfoxide in Human Plasma

An MRM LC-MS/MS method was developed to look for metopimazine sulfoxide (MPZS) in human plasma. The method was run in reverse phase in positive ion mode. The system was identical to what was described above except that a slightly longer column was used, a Supelco Discovery HS F5 100×4.0 mm (3 μm) column (Sigma-Aldrich, St. Louis, MO). A D6-isotopically labeled internal standards was used for MPS and the m/z transition monitored was 462.3→98.1. A calibration curve was generated for the quantification of MPZS in human plasma.

Data Analysis

The degree of disappearance of each analyte was evaluated by comparing the PAR of analyte to IS or concentration in different sampling time points to the PAR of time zero, (t=0, 100%). The percent of parent remaining values were calculated according the following equation:

% parent remaining=(mean concentration or PAR at each sampling time point/mean concentration or PAR of time zero)×100

All statistics (e.g., mean, SD) presented in the data tables are based on “precision as displayed” numbers (MS Excel, version 2010). GraphPad Prism (version 8, San Diego, CA) was used for figure generation.

Noncompartmental Pharmacokinetics analysis was performed in Phoenix 64 Winnonlin version 8.1.

Results

Clinical Pharmacokinetics

In vitro metabolism experiments had identified three potential metabolites, MPZA, MPZS and MPZH. Bioanalytical methods were developed to quantify MPZ, MPZA and MPZS in human plasma. The MPZH metabolite is isobaric with MPZS and it is separated enough from MPZS that it is visible in the same method in in vitro experiments (qualitative only). To look for the presence of these metabolites in vivo, plasma was sampled over 24 hours from 4 subjects from the highest SAD cohort. Following a single oral dose of 60 mg of metopimazine, the mean clinical exposure parameters are provided in Table 3.

TABLE 3
Mean pharmacokinetic exposure parameters of metopimazine and
its human metabolites following a single 60 mg clinical dose

| | | MPZ | | | MPZA | | | MPZS | | |
|---|---|---|---|---|---|---|---|---|---|---|
| | MPZ Dose | Cmax | Tmax | AUCₗₐₛₜ | Cmax | Tmax | AUCₗₐₛₜ | Cmax | Tmax | AUCₗₐₛₜ |
| Subject | (mg) | (ng/mL) | (hr) | (ng-h/mL) | (ng/mL) | (hr) | (ng-h/mL) | (ng/ml) | (hr) | (ng-h/mL) |
| 1401 | 60 | 38.9 | 3 | 111.9 | 241 | 3 | 1592.6 | 1.6 | 4 | 7.6 |
| 1404 | 60 | 203 | 0.75 | 278.2 | 601 | 1.25 | 2506.1 | 10.2 | 1.25 | 25.5 |
| 1405 | 60 | 75.6 | 0.75 | 129.4 | 357 | 1.25 | 1941.9 | 3.1 | 1 | 10.0 |
| 1408 | 60 | 58.2 | 1 | 167.5 | 390 | 1.5 | 2265.8 | 4.6 | 1.25 | 20.1 |

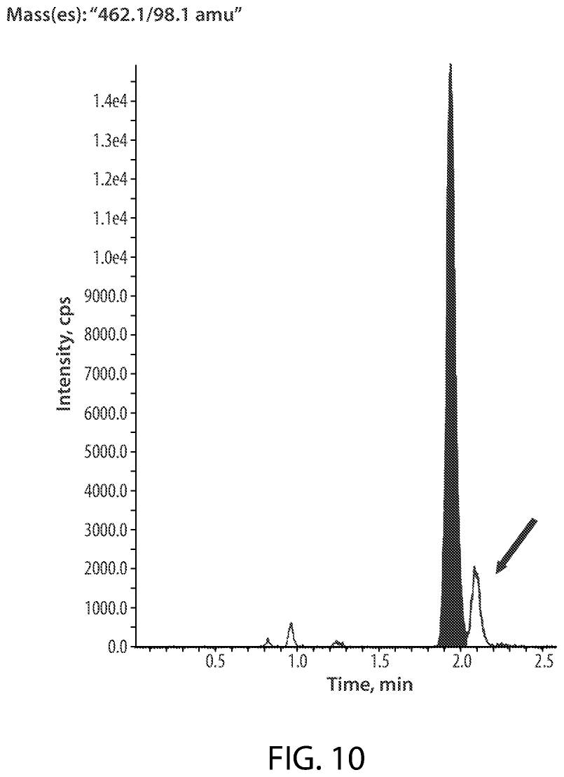

As expected, the major circulating metabolite was MPZA, with Tmax ranging from 1.25-3 hr and this analyte was present at all time points. MPZS was also found in all 4 subjects, though at much lower concentrations. It appeared at 30-45 min and was not quantifiable by 12 hours. A representative chromatogram is shown in FIG. 10. There was a very small isobaric peak that eluted just after 2 min (see arrow in FIG. 10) in some of the samples. While it is speculative, this could potentially be the MPZH metabolite, albeit present at only trace amounts and unlikely to be quantifiable even if a standard could be made. A rough estimate of percent circulating drug-related material of the verified in vivo metabolites based on AUClast is shown in Table 4.

TABLE 4
Percent drug-related material following a
single 60 mg clinical dose based on AUCₗₐₛₜ

| | |
|---|---|
| MPZ Dose | % Drug-related Material |

| Subject | (mg) | MPZ | MPZA | MPZS |
|---|---|---|---|---|
| 1401 | 60 | 6.5 | 93.0 | 0.4 |
| 1404 | 60 | 9.9 | 89.2 | 0.9 |
| 1405 | 60 | 6.2 | 93.3 | 0.5 |
| 1408 | 60 | 6.8 | 92.4 | 0.8 |

Based on this, MPZA accounts for approximately 90% of circulating material whereas the parent is less than 10%. Also, MPZS is a minor metabolite present at less than 1% of total circulating drug-related material.

Nonclinical Pharmacokinetics

Given that MPZA was the predominant circulating metabolite in humans, bioanalytical methods were developed to quantify both parent and the major human metabolite. To determine the pharmacokinetic properties of metopimazine and its acid metabolite in rats and dogs, single doses of metopimazine (free base) were administered orally to each species and plasma samples were taken over time and analyzed. A noncompartmental analysis of the bioanalytical data was performed and the pharmacokinetic parameters determined. Table 5 shows the mean PK exposure parameters in rats and dogs.

TABLE 5
Nonclinical pharmacokinetic exposure parameters
after a single dose of metopimazine

| Species | Metopimazine | | Cmax | Tmax | AUC_{inf} |
|---|---|---|---|---|---|
| (gender) | Dose (mg/kg) | Analyte | (ng/mL) | (h) | (ng-h/mL) |
| Rat | 45 | MPZ | 1080 | 4 | 8160 |
| (male) | | MPZA | 89 | 4 | 755 |
| Rat | 45 | MPZ | 1730 | 4 | 13800 |
| (female) | | MPZA | 106 | 4 | 944 |
| Dog | 10 | MPZ | 416 | 0.833 | 3190 |
| (male) | | MPZA | 103 | 2 | 1030 |

Interestingly, the MPZA metabolite to parent ratio is far lower in dogs and particularly lower in rats than in humans, indicating MPZA is disproportionately produced in humans.

While preferred embodiments of the present application have been shown and described herein, it will be obvious to those skilled in the art that such embodiments are provided by way of example only. Numerous variations, changes, and substitutions will now occur to those skilled in the art without departing from the application. It should be understood that various alternatives to the embodiments of the application described herein may be employed in practicing the application. It is intended that the following claims define the scope of the application and that methods and structures within the scope of these claims and their equivalents be covered thereby.

### INCORPORATION BY REFERENCE

All references cited in this application, and their references, are incorporated by reference herein in their entirety where appropriate for teachings of additional or alternative details, features, and/or technical background.

1. A process of preparing metopimazine acid, 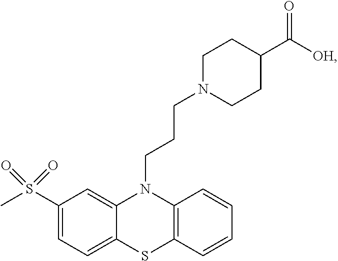 [SMILES: CS(=O)(=O)c1ccc2c(c1)N(CCCN1CCC(C(=O)O)CC1)c1ccccc1S2]
comprising contacting metopimazine, 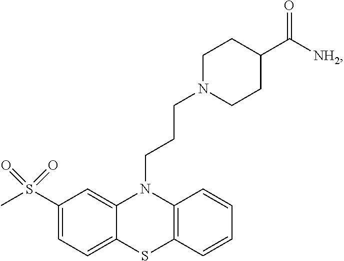 [SMILES: CS(=O)(=O)c1ccc2c(c1)N(CCCN1CCC(C(N)=O)CC1)c1ccccc1S2]
or a pharmaceutically acceptable salt thereof, with human microsomal liver amidase.

2. A process of preparing metopimazine acid, comprising contacting metopimazine, or a pharmaceutically acceptable salt thereof, with human liver cytosolic aldehyde oxidase.

3. The process of claim 1 or 2, wherein the metopimazine, or a pharmaceutically acceptable salt thereof, is metopimazine mesylate,

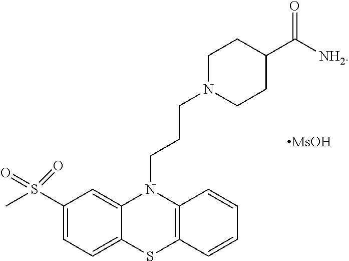 [SMILES: "·MsOH" ; CS(=O)(=O)c1ccc2c(c1)N(CCCN1CCC(C(N)=O)CC1)c1ccccc1S2]

4. A method of treating gastroparesis in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase.

5. A method of treating gastroparesis in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

6. The method of claim 4 or 5, wherein gastroparesis is diabetic gastroparesis.

7. The method of claim 4 or 5, wherein the gastroparesis is idiopathic gastroparesis.

8. The method of any of claims 4-7, wherein the gastroparesis comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation.

9. The method of claim 8, wherein the gastroparesis comprises the symptom nausea.

10. The method of claim 8, wherein the gastroparesis comprises the symptom vomiting.

11. A method of treating nausea associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase.

12. A method of treating nausea associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

13. A method of treating vomiting associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase.

14. A method of treating vomiting associated with gastroparesis in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

15. A method of improving gastric emptying in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase.

16. A method of improving gastric emptying in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

17. A method of treating functional and motility disorders of the GI tract in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase.

18. A method of treating functional and motility disorders of the GI tract in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

19. A method of treating an enteric nervous system disorder in a human subject in need thereof, comprising administering to the subject metopimazine, or a pharmaceutically acceptable salt thereof, in combination with an additional therapeutic agent, wherein the additional therapeutic agent is not metabolized by human microsomal liver amidase.

20. A method of treating an enteric nervous system disorder in a human subject in need thereof, comprising administering to the subject metopimazine, or a pharmaceutically acceptable salt thereof, in combination with an additional therapeutic agent, wherein the additional therapeutic agent is not metabolized by human liver cytosolic aldehyde oxidase.

21. The method of claim 19 or 20, wherein the enteric nervous system disorder is a chronic disorder.

22. The method of claim 19 or 20, wherein the enteric nervous system disorder is an acute disorder.

23. The method of claim 19 or 20, wherein the enteric nervous system disorder is selected from the group consisting of gastroparesis, Irritable Bowel Syndrome, lysosomal storage disorders, intestinal dysmotility, ganglioneuroma, multiple endocrine neoplasia type 2B (MEN2B), gastrointestinal neuropathy, functional dyspepsia, gastroesophageal reflux disease (GERD), and intestinal neuronal dysplasia.

24. The method of any of claims 19-23, wherein the enteric nervous system disorder comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation.

25. The method of claim 24, wherein the enteric nervous system disorder comprises the symptom nausea.

26. The method of claim 24, wherein the enteric nervous system disorder comprises the symptom vomiting.

27. The method of any one of claims 4-26, wherein the metopimazine, or a pharmaceutically acceptable salt thereof, is administered to the subject chronically.

28. The method of any one of claims 4-26, wherein the metopimazine, or a pharmaceutically acceptable salt thereof, is administered to the subject acutely.

29. The method of any one of claims 4-26, wherein the metopimazine, or a pharmaceutically acceptable salt thereof, is administered to the subject for at least 6 days.

30. The method of claim 29, wherein the metopimazine, or a pharmaceutically acceptable salt thereof, is administered to the subject for at least 7 days.

31. The method of claim 29, wherein the metopimazine, or a pharmaceutically acceptable salt thereof, is administered to the subject for at least four weeks.

32. The method of claim 29, wherein the metopimazine, or a pharmaceutically acceptable salt thereof, is administered to the subject for at least 12 weeks.

33. The method of any one of claims 4-32, wherein the metopimazine, or a pharmaceutically acceptable salt thereof, is administered to the subject one time per day.

34. The method of any one of claims 4-32, wherein the metopimazine, or a pharmaceutically acceptable salt thereof, is administered to the subject two times per day.

35. The method of any one of claims 4-32, wherein the metopimazine, or a pharmaceutically acceptable salt thereof, is administered to the subject three times per day.

36. The method of any one of claims 4-32, wherein the metopimazine, or a pharmaceutically acceptable salt thereof, is administered to the subject four times per day.

37. The method of any one of claims 4-36, wherein between about 5 mg and about 160 mg of the metopimazine, or a pharmaceutically acceptable salt thereof, is administered to the subject per day.

38. The method of any one of claims 4-36, wherein more than 20 mg of metopimazine, or a pharmaceutically acceptable salt thereof, is administered to the subject per day.

39. The method of any one of claims 4-38, wherein the metopimazine, or a pharmaceutically acceptable salt thereof, and the additional therapeutic agent are administered simultaneously in separate compositions.

40. The method of any one of claims 4-38, wherein the metopimazine, or a pharmaceutically acceptable salt thereof, and the additional therapeutic agent are administered at different times and in separate compositions.

41. The method of any one of claims 4-38, wherein the metopimazine, or a pharmaceutically acceptable salt thereof, and the additional therapeutic agent are administered in a composition in which both agents are present.

42. The method of any one of claims 4-41, wherein the metopimazine, or a pharmaceutically acceptable salt thereof, is suitable for administering orally, intraduodenally, intracolonically, parenterally, enterally, intraperitoneally, topically, transdermally, ophthalmically, intranasally, locally, non-orally, via spray, subcutaneously, intravenously, intratonsillary, intramuscularly, buccally, sublingually, intra-arterially, intrathecally, by infusion, by inhalation, or rectally.

43. The method of claim 42, wherein the metopimazine, or a pharmaceutically acceptable salt thereof, is suitable for administering orally, intraduodenally, intracolonically, enterally, topically, intranasally, non-orally, buccally, sublingually, by inhalation, or rectally.

44. The method of claim 43, wherein the metopimazine, or a pharmaceutically acceptable salt thereof, is suitable for administering orally.

45. The method of claim 43, wherein the metopimazine, or a pharmaceutically acceptable salt thereof, is suitable for administering sublingually.

46. The method of any one of claims 4-41, wherein the metopimazine, or a pharmaceutically acceptable salt thereof, is formulated as a tablet, a capsulean oil, a capsule, a gel, a paste, a powder, a suspension, a syrup, an enema, a suppository, an emulsion, a solution, an extended-release formulation, or a modified-release formulation.

47. The method of claim 46, wherein the metopimazine, or a pharmaceutically acceptable salt thereof, is formulated as a tablet, a capsule, a paste, a powder, a suspension, a suppository, an extended-release formulation, or a modified-release formulation.

48. The method of claim 47, wherein the metopimazine, or a pharmaceutically acceptable salt thereof, is formulated as an extended-release formulation.

49. The method of claim 47, wherein the metopimazine, or a pharmaceutically acceptable salt thereof, is formulated as a capsule.

50. The method of any one of claims 4-49, wherein the metopimazine, or a pharmaceutically acceptable salt thereof, is metopimazine mesylate.

51. A pharmaceutical composition comprising metopimazine, or a pharmaceutically acceptable salt thereof, in combination with an additional therapeutic agent, wherein the additional therapeutic agent is not metabolized by human microsomal liver amidase.

52. A pharmaceutical composition comprising metopimazine, or a pharmaceutically acceptable salt thereof, in combination with an additional therapeutic agent, wherein the additional therapeutic agent is not metabolized by human liver cytosolic aldehyde oxidase.

53. The pharmaceutical composition of claim 51 or 52, wherein the metopimazine, or a pharmaceutically acceptable salt thereof, is a pharmaceutically acceptable salt of metopimazine.

54. The pharmaceutical composition of claim 53, wherein the pharmaceutically acceptable salt of metopimazine is metopimazine mesylate.

55. A method of treating gastroparesis in a human subject in need thereof, comprising administering to the subject a pharmaceutical composition of any of claims 51-54.

56. The method of claim 55, wherein gastroparesis is diabetic gastroparesis.

57. The method of claim 55, wherein the gastroparesis is idiopathic gastroparesis.

58. The method of any of claims 55-57, wherein the gastroparesis comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation.

59. The method of claim 58, wherein the gastroparesis comprises the symptom nausea.

60. The method of claim 58, wherein the gastroparesis comprises the symptom vomiting.

61. A method of treating nausea associated with gastroparesis in a human subject in need thereof, comprising administering to the subject a pharmaceutical composition of any of claims 51-54.

62. A method of treating vomiting associated with gastroparesis in a human subject in need thereof, comprising administering to the subject a pharmaceutical composition of any of claims 51-54.

63. A method of improving gastric emptying in a human subject in need thereof, comprising administering to the subject a pharmaceutical composition of any of claims 50-53.

64. A method of treating functional and motility disorders of the GI tract in a human subject in need thereof, comprising administering to the subject a pharmaceutical composition of any of claims 51-54.

65. A method of treating an enteric nervous system disorder in a human subject in need thereof, comprising administering to the subject a pharmaceutical composition of any of claims 51-54.

66. The method of claim 65, wherein the enteric nervous system disorder is a chronic disorder.

67. The method of claim 65, wherein the enteric nervous system disorder is an acute disorder.

68. The method of claim 65, wherein the enteric nervous system disorder is selected from the group consisting of gastroparesis, Irritable Bowel Syndrome, lysosomal storage disorders, intestinal dysmotility, ganglioneuroma, multiple endocrine neoplasia type 2B (MEN2B), gastrointestinal neuropathy, functional dyspepsia, gastroesophageal reflux disease (GERD), and intestinal neuronal dysplasia.

69. The method of any of claims 65-68, wherein the enteric nervous system disorder comprises a symptom selected from the group consisting of early satiety, post-prandial fullness, abdominal fullness, nausea, vomiting, delayed gastric emptying, diarrhea, abdominal pain, gas, bloating, gastroesophageal reflux, reduced appetite, and constipation.

70. The method of claim 69, wherein the enteric nervous system disorder comprises the symptom nausea.

71. The method of claim 69, wherein the enteric nervous system disorder comprises the symptom vomiting.

72. The method of any one of claims 55-71, wherein the pharmaceutical composition is administered to the subject chronically.

73. The method of any one of claims 55-71, wherein pharmaceutical composition is administered to the subject acutely.

74. The method of any one of claims 55-71, wherein the pharmaceutical composition is administered to the subject for at least 6 days.

75. The method of claim 74, wherein the pharmaceutical composition is administered to the subject for at least 7 days.

76. The method of claim 74, wherein the pharmaceutical composition is administered to the subject for at least four weeks.

77. The method of claim 74, wherein the pharmaceutical composition is administered to the subject for at least 12 weeks.

78. The method of any one of claims 55-77, wherein the pharmaceutical composition is administered to the subject one time per day.

79. The method of any one of claims 55-77, wherein the pharmaceutical composition is administered to the subject two times per day.

80. The method of any one of claims 55-77, wherein the pharmaceutical composition is administered to the subject three times per day.

81. The method of any one of claims 55-77, wherein the pharmaceutical composition is administered to the subject four times per day.

82. The method of any one of claims 55-81, wherein between about 5 mg and about 160 mg of the metopimazine, or a pharmaceutically acceptable salt thereof, is administered to the subject per day.

83. The method of any one of claims 55-81, wherein more than 20 mg of metopimazine, or a pharmaceutically acceptable salt thereof, is administered to the subject per day.

84. The method of any one of claims 55-83, wherein the pharmaceutical composition is suitable for administering orally, intraduodenally, intracolonically, parenterally, enterally, intraperitoneally, topically, transdermally, ophthalmically, intranasally, locally, non-orally, via spray, subcutaneously, intravenously, intratonsillary, intramuscularly, buccally, sublingually, intra-arterially, intrathecally, by infusion, by inhalation, or rectally.

85. The method of claim 84, wherein the pharmaceutical composition is suitable for administering orally, intraduodenally, intracolonically, enterally, topically, intranasally, non-orally, buccally, sublingually, by inhalation, or rectally.

86. The method of claim 84, wherein the pharmaceutical composition is suitable for administering orally.

87. The method of claim 84, wherein the pharmaceutical composition is suitable for administering sublingually.

88. The method of any one of claims 55-81, wherein the pharmaceutical composition is formulated as a tablet, a capsulean oil, a capsule, a gel, a paste, a powder, a suspension, a syrup, an enema, a suppository, an emulsion, a solution, an extended-release formulation, or a modified-release formulation.

89. The method of claim 88, wherein the pharmaceutical composition is formulated as a tablet, a capsule, a paste, a powder, a suspension, a suppository, an extended-release formulation, or a modified-release formulation.

90. The method of claim 88, wherein pharmaceutical composition is formulated as an extended-release formulation.

91. The method of claim 88, wherein pharmaceutical composition is formulated as a capsule.
